(19) United States
(12) Patent Application Publication
(10) Pub. No.: US 2015/0353912 A1
(43) Pub. Date: Dec. 10, 2015

(54) METHODS FOR TREATING AND PREVENTING RADIATION INJURY USING ACTIVATED PROTEIN C POLYPEPTIDES

(71) Applicants: Hartmut Weiler-Guettler, Delafield, WI (US); John Henry Griffin, De Mar, CA (US); Hartmut Geiger, Ulm (DE); Martin Kristian Hauer-Jensen, Little Rock, AR (US); Laurent Olivier Mosnier, San Diego, CA (US)

(72) Inventors: Hartmut Weiler-Guettler, Delafield, WI (US); John Henry Griffin, De Mar, CA (US); Hartmut Geiger, Ulm (DE); Martin Kristian Hauer-Jensen, Little Rock, AR (US); Laurent Olivier Mosnier, San Diego, CA (US)

(73) Assignee: CHILDREN'S HOSPITAL MEDICAL CENTER, Cincinnati, OH (US)

(21) Appl. No.: 14/403,658

(22) PCT Filed: May 30, 2013
(86) PCT No.: PCT/US2013/043264

§ 371 Date: Nov. 25, 2014

Related U.S. Application Data
(60) Provisional application No. 61/653,903, filed on May 31, 2012.

(51) Int. Cl. C12N 9/64; A61K 45/06; A61K 38/48

(52) U.S. Cl. CPC C12N 9/6464; A61K 38/4866; A61K 45/06; C12Y 304/21069

(57) ABSTRACT

Methods of treating and preventing radiation injury are provided by the present invention. In particular, provided herein are methods comprising administering to a subject an Activated Protein C (APC), Plasma Zymogen Protein C (PC), or a variant thereof to treat or prevent radiation injury and to reduce chemical toxicity in subjects receiving myelosuppressive therapy.

### CROSS-REFERENCE TO RELATED APPLICATIONS

This application claims the benefit of U.S. Provisional Application No. 61/653,903, filed on May 31, 2012, which is incorporated by reference herein in its entirety.

### STATEMENT REGARDING FEDERALLY SPONSORED RESEARCH

This invention was made with government support under HL087618, CA71382, AI67798, AI080557, HL31950, HL052246, HL44612, and CA122023 awarded by the National Institutes of Health. The government has certain rights in the invention.

### FIELD OF THE INVENTION

The present invention provides methods of treating radiation injury. In particular, the present invention provides methods of using a polypeptide of Activated Protein C (APC), Plasma Zymogen Protein C (PC), and variants thereof for treating radiation injury, for preventing radiation injury, and for reducing chemical toxicity in a subject receiving, for example, ionizing radiation and/or chemotherapy.

### BACKGROUND

Exposure to ionizing radiation causes injury to many systems of the body, most notably rapidly dividing cells present in the bone marrow and gut. Injuries to the latter target organs are the primary survival determinants after life-threatening radiation exposure. Exposure to small or moderate radiation doses causes a profound decrease of cells in the bone marrow that places patients at risk of death from bleeding (secondary to thrombocytopenia) and from infection (secondary to neutropenia). Research for developing radioprotective protocols has focused primarily on protecting against cell damage using antioxidant therapy, suppressing radiation induced apoptosis and other forms of cell death, and using cytokines to enhance tissue regeneration post-exposure. To date, few effective clinical therapies exist that mitigate the effects of total body irradiation (TBI) on hematological or gastrointestinal toxicities, particularly when treatment can only be initiated post-exposure as is typical in the context of a radiological emergency or disaster. Accordingly, there remains a need for effective prophylactic and post-exposure treatments for patients receiving intense radiation treatments and for victims of nuclear accidents and other intentional or accidental radiation exposure events.

### SUMMARY OF THE INVENTION

In one aspect, the present invention provides a method of treating a radiation injury in a subject. In one embodiment, the method comprises administering an effective amount of a polypeptide of Activated Protein C (APC), Plasma Zymogen Protein C (PC) or variants thereof to a subject. As used herein, the term “APC, PC or variant thereof” refers to a wild-type APC polypeptide, an APC polypeptide variant that comprises at least one amino acid substitution relative to full-length wild-type, a full-length wild-type PC polypeptide, a PC polypeptide variant, that comprises at least one amino acid substitution relative to a full-length wild-type PC polypeptide, or a truncated, rearranged or modified molecule of any of the aforementioned molecules. In some cases, an effective amount of an APC polypeptide variant is smaller or larger than the effective amount of wild-type APC polypeptide to provide comparable treatment or prevention of radiation injury. A large number of APC and PC variants exist each with specific bioactivity for various cell-protective effects. APC, PC, and variants thereof demonstrate radioprotective effects for subjects exposed to lethal or sub-lethal doses of radiation. The radioprotective effect is predicted to apply to other forms of toxicity, to the rapidly dividing cells of the bone marrow and gut, including chemical toxicity like that found during chemotherapy.

APC, PC, and some variants thereof can be administered prior to, immediately prior to, concurrently with, or following exposure of the subject to radiation. The radiation can comprise whole body irradiation. The radiation can be ionizing radiation. The ionizing radiation can be cosmic radiation, nuclear medicine, x-rays, nuclear fuel-derived, or be associated with nuclear fallout. The radiation can be non-ionizing radiation. Exposure of the subject to radiation can comprise brachytherapy. Brachytherapy can be performed in combination with surgery, external radiation therapy, or chemotherapy. Only a portion of the subject can be exposed to radiation. The method can further comprise administering an effective amount of an anti-oxidant to the subject. The anti-oxidant can be γ-tocotrienol. The method can further comprise administering an effective amount of a TLR-5 agonist drug to the subject. The TLR-5 agonist can be CBLB502 or other TLR-5 agonists.

Administering can comprise administering an effective amount of APC, PC, or a variant thereof as described in FIG. 14. APC, PC, or a variant thereof can be given intravenously or by intraperitoneal injection. APC, PC, or a variant thereof can be administered in a bolus or by continuous infusion. Bolus dosing can comprise administering a single dose or two or more doses of the polypeptide. The subject can be a human. The subject can be a non-human animal. The non-human animal can be a livestock animal.

### BRIEF DESCRIPTION OF THE DRAWINGS

FIG. 1 presents graphs and images demonstrating that elevated Thbd expression selects for primitive hematopoietic cells upon irradiation in vivo. (a) EGFP chimerism in peripheral blood (PB) of Animal 9 after exposure to 3 Gy TBI every week for 3 consecutive weeks versus controls (same experiment, non-selected). (b) Graphical representation of the provirus integration into chromosome 2 of bone marrow (BM) cells of Animal 9 as determined by LM-PCR followed by sequencing of the dominant integration product. See FIG. 15, which sets forth all integration sites in Animal 9. (c) Transcriptional level of expression of the genes surrounding 5′ and 3′ side of integrated provirus of Animal 9 (BM cells). Determination by quantitative real-time RT-PCR compared to a pool of control samples (BM cells from C57BL/6CD45.1 mice, and Animals 11 and 18, which were transplanted with transduced HSPCs but not irradiated). (d) Expression of Thbd in the BM of Animal 9 compared to control animals (C57BL/6), determined by Western blot analysis. (e) Experimental setup: over-expression of Thbd in HSPCs by retroviral transduction, followed by transplantation and subsequent selection by irradiation. Recipient animals were pre-conditioned by irradiation with 11.75 Gy to accept the graft. Animals consistently presented with a graft chimerism (CD45.2⁺ cells in PB) of higher than 90% 3 weeks post-transplantation. (f) Representative data depicting the level of EGFP expression/selection among CD45.2⁺ cells in PB of individual animals with and without irradiation in Thbd-transduced compared to control-transduced cells transplanted. (g) Quantification of the radioselection of Thbd-transduced hematopoietic cells in PB in vivo post-irradiation relative to control-transduced (GFP only) hematopoietic cells; n=3 independent experiments with at least 3 recipients per single experiment. *p=<0.05.

FIG. 2 presents graphs and schematic demonstrating radiation toxicity mitigation with Solulin and recombinant aPC. (a, b) 30 day survival of mice injected subcutaneously (n=8 per group) 30 minutes after exposure to (a) 8.5 Gy or (b) 9.5 Gy TBI, p<0.05 for both Solulin at 3 mg kg⁻¹ and control. (c) Experimental setup for experiments depicted in (d). d) 30 day survival of C57BL/6 animals injected with recombinant murine aPC (0.35 to 0.4 mg kg⁻¹) or vehicle (PBS) i.v. 30 minutes after single-dose TBI; n=8 for 8 Gy vehicle, n=33 for 9 Gy aPC, n=23 for 9 Gy vehicle, n=8 for 10 Gy vehicle, n=20 for 10 Gy multiple aPC (30 min, 1 h, and 2 h post-irradiation, 9 Gy vehicle vs. 9 Gy aPC, p<0.0001; 10 Gy vehicle vs. 10 Gy multiple aPC, p<0.0001). (e) Experimental setup: mitigation of TBI toxicity when aPC is given no earlier than 24 hours post-TBI. (f) 30 day survival of C57BL/6 animals injected with vehicle or murine aPC at a dose of 5 μg/mouse through a single i.v. injection at 24- and 48-hours post TBI (9.5 Gy).

FIG. 3 presents data suggesting a mode of action for radiation mitigation by soluble Thbd and aPC. (a) Experimental design. (b) Total number of leukocytes per femur in control animals and animals treated with aPC 10 days post-irradiation. (c) Frequency of Lin−, c-Kit+ cells in BM 10 days post-irradiation. (d) Frequency of CFCs in BM cells 10 days post-irradiation. (e) Experimental design for data presented in s(f). (f) Frequency of CFCs in BM cells 10 days post irradiation. (g) 30 day survival of mice (n=15 per group) irradiated with an LD₅₀ receiving either 5 μg/mouse of the hyper-anticoagulant E149A mutant form of aPC or vehicle control through a single tail vein injection 30 min after TBI, p<0.001 for E149A-aPC as well as for aPC versus saline and 5A-aPC. (h) 30 day survival of mice given a LD_{50/30} TBI and treated with the histone blocking antibody BWA3 (i.p. 30 mg kg⁻¹) at 0 hours (black lines) or 16 hours (green lines) after irradiation, as compared to control injections of IgG Ab, n≧12 per experimental group. (i) 30 day survival of mice given a LD_{50/30} TBI and treated with an anti-factor XIa antibody (14E11) at 0 hours post-irradiation (i.p. 30 mg kg⁻¹) as compared to control Ab treated animals, n≧0.16 per experimental group.

FIG. 4 presents graphs, images, and schematics for assessing the role of endogenous Thbd in radiation protection. (a) Expression of Thbd relative to β-actin in hematopoietic cells. As an additional control, RNA from BM cells from a floxed Thbd allele crossed to an Mx-Cre animals treated with pIC to delete the allele in BM cells (601 BM, deletion of up to 80% confirmed by PCR) was used. (b) Expression of Thbd relative to β-actin in bone marrow stromal cells containing endothelial cells (CD45−, Ter119− cells) and in non-endothelial stromal cells (CD45−, Ter119−, CD31− cells). (c) Femoral bone marrow of Thbd^{Pro/lacZ} mice stained in situ with a substrate for β-galactosidase. Blue staining indicates expression of the LacZ reporter gene controlled by the endogenous Thbd-promoter. (left) Staining occurs in the endothelium of blood vessel on the outer surface of the bone or penetrating the bone (black arrowhead), and in some vessels within the bone marrow mass. Intense staining is seen in a loose meshwork of cells located between the entire inner surface of the bone and the central bone marrow (white arrow; BM: bone marrow partially exposed by removal of the lacZ-positive layer). No staining was noted in parallel-processed WT controls. (middle) LacZ-positive clusters of cells are predominantly associated with the periphery of the marrow extruded from the bone cavity. (right) lacZ-positive endothelial cells seen in small blood vessels supplying the outer bone surface. The expression of lacZ correlated well with the data obtained by real-time RT-PCR in (a, b). White bar represents 1 mm. (d) 30 day survival of Thbd^{+/+} and Thbd^{Pro/lacZ} mice (n=8 per group) subjected to TBI with doses ranging between 7-11 Gy, p=0.0001. (e) 20 day survival of Thbd^{Pro/Pro} animals irradiated with an LD_{50/30} TBI, n≧11 per group, p=0.09. (f) 30 day survival of animals treated with an LD_{50/30} TBI, n=10 for APC^{HI}, n=15 for Thbd^{Pro/Pro}/APC^{HI} and WT control, p<0.05 for Thbd^{Pro/Pro}/APC^{HI} over WT. (g) Experimental setup: competitive transplantation/radioselection experiments with BM cells from Thbd^{Pro/Pro} mice. (h) Donor chimerism in peripheral blood (PB) of animals competitively transplanted according to (g) 3 weeks post-3 Gy irradiation of recipients compared to controls, n≧4 recipients per group, *=p<0.05. (i) Experimental setup: transplantation/radioselection experiments with Thbd^{Pro/Pro} and WT mice reconstituted with WT bone marrow. (j) 30 day survival of WT and Thbd^{Pro/Pro} recipients treated with a second dosage of 7.75 Gy, n=20 for WT and n=9 for Thbd^{Pro/Pro} recipients, p<0.05 for Thbd^{Pro/Pro} recipients compared to WT recipients.

FIG. 5 presents data demonstrating that retroviral insertional mutagenesis with replication-deficient retroviridae permits identification of integration sites in primitive hematopoietic cells that confer positive selection upon total body irradiation (TBI). (a) Experimental setup for identification procedure. (b) Relative contribution of cells transduced with retrovirus (EGFP+ cells) as percentage of donor cells (Ly5.2 cells) upon transplantation and DNA damage in vivo (animals 7, 8 and 9) compared to the relative contribution of non-irradiated animals (animals 10 and 11). One out of four independent experiments is shown. (c) Quantification of the enrichment of tranduced (EGFP+) cells in PB upon DNA-damage induced by irradiation with 3Gy compared to transduced but not selected animals from experiment depicted in FIG. 1B. (d) Fluorescence histogram of PB cells selected post-irradiation in vivo (14 weeks post initial transplant) compared to control (non-irradiation selected cells). The distribution of the EGFP signal as depicted in the histogram in animals 7 to 9 compared to the controls (Animals 10 and 11) indicates at least oligoclonal selection of primitive hematopoietic cells upon DNA damage in vivo (individually defined peaks in Animals 7-9). (e) Representative retroviral integration sites in BM cells derived from Animals 7 to 11 detected by LM-PCR, upon sequencing of the band cut from the gel. The gene name lists the gene closest to the integration site of the provirus. See FIG. 15, which sets forth all integration sites identified in radio-selected BM in our experiments. Shown are mean values+1 SEM.

FIG. 6 presents data demonstrating the effects of integration at the PUMA locus. (a) EGFP chimerism in peripheral blood (PB) of Animal 2 relative to controls upon DNA damage selection. (b) Graphical representation of the integration of the provirus in BM cells from Animal 2 as determined by LM-PCR followed by sequencing of the integron product for the dominant integration site. See FIG. 15, which sets forth all integration sites in Animal 2. Note that the provirus integrated into intron 2 of PUMA. (c) Expression of the genes 5′ and 3′ from the integration site determined by quantitative real-time PCR as compared to a pool of control samples (C57BL/6, CD45.1+, BM from Animals 11 and 18, transduced and transplanted but not selected by DNA damage). (d) Elevated expression of PUMA in cells from BM and spleen from Animal 2 compared to C57BL/6 control as determined by Western Blot. Note that only in tissue from Animal 2 is a second, much larger unidentified specific form/variant of PUMA detected. This is likely not a PUMA-EGFP fusion protein, as EGFP can be detected in Animal 2 at the expected size, and the alternate PUMA band is much larger than a putative PUMA-EGFP fusion gene. Shown are mean values+1 SEM.

FIG. 7 presents an image and a set of data plots as verification the Thbd expression system in transduced cells. (a) Detection of Thbd in non-transduced 3T3 cells and 3T3 cells transduced with either Thbd vector or SFB91 control by Western Blot. Mouse lung lysate served as a positive control, μg=μg of protein loaded on the gel. (b) Detection of Thbd on EGFP positive transduced hematopoietic cells by flow cytometry with an anti-Thbd AB before transplantation versus EGFP control transduced cells. While BM cells express to a certain extent Thbd (EGFP+ control transduced cells, see also FIG. 5A), almost all cells that were transduced with the Thbd vector (EGFP+ and thus Thbd transduced) express Thbd on the cell surface.

FIG. 8 is a series of graphs demonstrating that intrinsically elevated levels of Thbd in hematopoietic progenitor cells do not confer cell-intrinsic protection from radiation toxicity. HPCs tranduced with the Thbd expression vector were subjected to various assays to determine radiation response in vitro. (a) Expression of Thbd does not alter colony-forming cell activity. (b) Survival of CFC post-irradiation as well as the percentage of apoptotic cells upon irradiation of control and Thbd transduced HPCs (c). (d) Proliferative expansion/differentiation of control and Thbd-transduced HPCs post-irradiation. Shown are mean values+1 SEM. While radiation significantly interfered with these parameters (p<0.05), there were no significant differences between the response of the control and the Thbd transduced HPCs in these experiments.

FIG. 9 presents data to demonstrate that solulin shows superior resistance to ionizing radiation with regard to activation of PC as compared to a similar, but non-mutated form of recombinant Thbd. The ability to act as a cofactor for PC activation was determined at thrombomodulin/solulin concentrations of 2.5 nM after exposure to increasing doses of radiation (0 to 20 Gy). The graph shows generation of APC over a 60 minute period relative to thrombin, protein C, and buffer alone, in absolute values (A) and normalized to unirradiated samples (B). Shown is a representative experiment done in triplicate. Solulin exhibits significantly increased resistance to inactivation by radiation relative to non-mutated thrombomodulin (p<0.0001). The difference in potency between the two thrombomodulin variants is attributed to solulin lacking a chondroitin sulfate side chain.

FIG. 10 presents data to demonstrate that Intrinsically elevated levels of Thbd in hematopoietic progenitor cells do not confer cell-intrinsic protection from radiation toxicity. HPCs tranduced with the Thbd expression vector were subjected to various assays to determine radiation response in vitro. (a) Expression of Thbd does not alter colony-forming cell activity. (b) Survival of CFC post irradition as well as the percentage of apoptotic cells (c) upon irradiation of control and Thbd transduced HPCs. (d) Proliferative expansion/differentiation of control and Thbd-transduced HPCs post irradiation. Shown are mean values+1 SEM. While radiation significantly interfered with these parameters (p<0.05), there were no significant difference between the response of the control and the Thbd transduced HPCs in these experiments.

FIG. 11 demonstrates selection of PAR1^{-/-} and EPCR^{LOW} hematopoietic cells upon irradiation. (a) Experimental setup of the competitive transplants/radioselection experiments to determine radioselection of PAR1^{-/-} and EPCR low hematopoietic cells. Donor chimerism in PB 3 weeks post 3Gy irradiation in vivo, normalized to the non-irradiated control group of (b) C56BL/6 and PAR1^{-/-} hematopoietic cells and (c) C57BL/6 and EPCR^{-/pro} (EPCR low) hematopoietic cells. n=at least 10 recipients for each experimental group, *=p<0.05. Shown are mean values+1 SEM.

FIG. 12 is a set of graphs presenting biomarkers indicative of the activation state of the blood coagulation system in PB in response to exposure to TBI. Surrogate plasma markers of coagulation activation, i.e., thrombin-antithrombin complex (TAT) and the fibrin degradation product D-dimer were determined in PB upon TBI with a LD₅₀. Neither parameter was unaltered over a 24 hour period following exposure, a timeframe during which the animals are responsive to aPC therapy, indicating that radiation exposure does not trigger a systemic activation of coagulation, but that such effects (if they occur) are likely highly localized.

FIG. 13 is a series of plots demonstrating expression of Thbd in bone marrow cells. (a) Thbd is detected by flow cytometry in Ly-6G-negative/Gr1-CD115-positive macrophages (gate A; upper panel), as well as in B220 positive B-cells. (b) Thbd-expressing macrophages (gate B, panel d) are distinct from the two previously described populations of BM resident macrophage-like cells involved in maintenance of the hematopoietic niche in BM, i.e., cells with the surface phenotype CD169POS CD115INT F4/80POS Gr1NEG or CD11bPOS Ly-6GPOS F4/80POS. (c) Thbd is detected by flow cytometry in CD45NEG Ter119NEG non-hematopoietic BM stromal endothelial cells expressing CD31 (gate A; histogram A). Within the endothelial population, Thbd expression is restricted to Sca-1-negative sinusoidal endothelium (histogram/gate B); but is absent from Sca-1-expressing arterial endothelium (histogram/gate C).

FIG. 14 is a table presenting dosing quantities and ranges of APC and PC and variants thereof which are predicted to be an effective amount of polypeptide which when delivered to a subject would protect the subject from radiation injury, chemical injury, or myeloabalation.

FIG. 15 is a table setting forth all virus integration sites detected in the genomic DNA of bone marrow cells obtained from animals that received post-transplant total body irradiation.

FIG. 16 sets for human protein C amino acid sequence (SEQ ID NO:1). Numbering below the sequence represent that of the mature protein; numbering above the sequence indicates numbering according to alternative chymotrypsin nomenclature equivalents. Active site residues are indicated by stars. Drawing adapted from Wildhagen et al., Thromb. Haemost. 106:1034-1045 (2011).

### DETAILED DESCRIPTION OF THE INVENTION

Generally:

The present invention is based, at least in part, on the inventors' discovery that Activated Protein C (APC), recombinant APC polypeptide variants, and precursor molecules like Protein C or their variants have protective properties with respect to injury caused by chemicals or radiation. In particular, the inventors discovered that the administration of APC or a recombinant APC variant having specifically altered properties significantly reduces mortality caused by whole body exposure to otherwise lethal doses of ionizing radiation. The life-saving effect is observed even when APC or PC polypeptide is administered as late as 24 hours after the exposure and occurs in the absence of other supportive treatment. The inventors also discovered that this effect of APC or PC polypeptide can be recapitulated by recombinant variants that have been engineered for enhanced potency, enhanced bioactivity, or enhanced latent bioactivity that is unmasked upon thrombomodulin-independent activation of Plasma Zymogen Protein C (PC) (e.g., a “hyperactivatable” variant of PC).

Biology:

APC, a trypsin-like serine protease with a typical active site triad (Ser360, His211, and Asp257), is generated by thrombin's cleavage of PC in the presence of thrombomodulin (Thbd) and endothelial protein C receptor (EPCR). The APC protease domain (residues 170-419 of human APC) is homologous to thrombin and Factors Xa, IXa, and Vila, each of which contains similar trypsin-like active sites. APC and its cofactors from the Plasma Zymogen Protein C pathway have two major functions: anticoagulant activity and cytoprotective activity. For review, see, e.g., Mosnier & Griffin, Front. Biosci. 11:2381-99 (2006). Such activities suggest that APC and its cofactors have important roles in the body's host-defense system. APC has been shown to reduce sepsis mortality in animal models and in human patients and was FDA-approved in 2001 for clinical use in the treatment of severe sepsis. Bernard et al., N. Engl. J. Med. 344(10):759-62 (2001); Taylor et al., J. Clin. Invest. 79(3):918-925 (1987).

Methods Involving APC or PC Polypeptides:

The present invention provides methods of treating or preventing radiation injury or chemical toxicity in a subject. In some cases, the method comprises administering an effective amount of APC, PC, or a variant thereof to a subject exposed to radiation or a toxic chemical. As used herein, “APC, PC or variant thereof” refers to wild-type polypeptides of APC or PC and to variants comprising at least one amino acid modification relative to the amino acid sequence of an unmodified polypeptide (i.e., wild-type APC, wild-type PC) or relative to the amino acid sequence of a truncated or otherwise modified form of APC or PC. For example, an APC polypeptide variant can comprise at least one amino acid residue modification relative to a full-length, wild-type human APC polypeptide having the amino acid sequence set forth as SEQ ID NO:1. A modification can comprise substitution of the serine residue at position 11 for glycine, substitution of the glutamine at position 32 with a glutamic acid residue, substitution of the glutamic acid at position 149 with an alanine, or substitution of the asparagine at position 33 with an aspartic acid residue, where the amino acid residue positions are numbered relative to SEQ ID NO:1. A variant form can also take the form of any amino acid substitution at the aforementioned positions of 11, 32, 33, 149, 167, or 172 wherein the amino acid substitution is any amino acid other than that of the wild-type molecule. Other variant forms could be produced by one of skill in the art that modified the wild-type APC or PC sequence. In some cases, an APC polypeptide variant can comprise a mutation as previously described by Wildhagen et al., Thromb Haemost 106:1034-1045 (2011) or Griffin et al. Int J Hematol 95:333-345 (2012). A Plasma Zymogen Protein C variant can be hyperactivatable relative to wild-type PC. As used herein, the term “hyperactivatable” refers to the ability of a Plasma Zymogen Protein C polypeptide to be efficiently activated by thrombin in the absence or presence of various known cofactors. In some cases, a hyperactivatable PC polypeptide can comprise substitutions that replace aspartic acid residues at positions 167 and 172 with a phenylalanine residue and a lysine residue, respectively, where the amino acid positions are numbered relative to a wild-type PC polypeptide having the amino acid sequence as set forth in SEQ ID NO:1. In some cases, additional molecular modifications to the core APC or PC polypeptide can be made to enhance targeting or half-life of the molecule or to add some relevant biological feature. Examples of such modifications include glycosylation, pegylation, addition of receptors or peptides to target specific cell types or locations within cells, post-translational modifications, oxidative modifications like carbonylation or disulfide formation and others. A skilled artisan will understand that polypeptide sequences presented herein can vary somewhat, whether as a result, e.g., of sequencing error or allelic variation or duplication, from the sequence presented while still retaining their essential nature. Because of the degeneracy in the genetic code, the polypeptide sequences disclosed can also be encoded by a variety of polynucleotide sequences, all of which are within the scope of the invention. Polypeptides of the invention include polymorphic variants, alleles, mutants, and interspecies homologs.

APC, PC, or a variant thereof can be isolated from a natural source or be synthesized. One of skill in the art recognizes the advantageous efficiency of producing recombinant proteins, rather than isolating proteins from an animal source, to improve efficiency and to minimize possible animal protein contamination.

In some cases, APC, PC, or a variant thereof are produced by introducing a vector encoding at least one polypeptide described herein into a host cell capable of expressing the encoded variant on the vector to produce the protein. The vector includes a sequence encoding at least one APC, PC, or variant polypeptide, where the sequence can be operably linked to an expression control element or expression control sequence for expression in the host cell. Expression control elements or sequences appropriate for the methods provided herein can include, without limitation, promoters (e.g., transcriptional promoters), enhancers, and upstream or downstream untranslated sequences. Suitable host cells include bacterial cells, such as E. coli, and eukaryotic cells, such as yeast cells, insect cells, avian cells, or mammalian cells. The polypeptide can then be isolated from the host cells by any method suitable for recovering functional protein.

Types of Injury and Treatment:

In one aspect, the present invention provides methods for treating radiation injury in a subject. As used herein, the term “radiation injury” refers to any type of tissue damage resulting from exposure to radiation (e.g., ionizing radiation, non-ionizing radiation). Radiation injury can include cutaneous radiation injury (i.e., injury to the skin and underlying tissues from acute exposure to radiation) and acute radiation syndrome (i.e., serious illness that occurs following an acute high dose of penetrating whole body irradiation). Symptoms associated with radiation injury include nausea, vomiting, diarrhea, loss of appetite, fatigue, fever, hair loss, infection, hematologic toxicity (e.g., thombocytopenia, leucopenia, anemia), swelling, itching, and redness of the skin, and, in severe cases, seizures, coma, and death. Tissues with rapid cell turnover (e.g., skin, stomach and intestinal lining, bone marrow) are most susceptible to radiation injury. In some cases, a radiation injury is associated with total body irradiation (TBI). TBI typically involves irradiation of the entire body although, in some cases, the lungs or other tissues may be at least partially shielded from irradiation to reduce the risk of radiation-induced injury to those specific tissues. Doses of total body irradiation can vary. For example, radiation doses used in murine bone marrow transplantation typically range from about 10 to greater than 12 Gy. Frequently, such high doses are achieved by spreading the total dose out between several sessions of exposure, often with an interval of time in between exposures. Lower doses of radiation in the range of one to ten Gy can be used to condition a subject in a non-lethal manner, causing injury and toxicity to cells of the bone marrow and gut.

As used herein, the terms “treating” and “to treat” refer to improving, reducing, eliminating, mitigating, or lessening the severity of any aspect of radiation injury in a subject. In some cases, treating radiation injury can include reducing mortality, improving regeneration of damaged tissues, and promoting survival in a subject receiving radiation therapy or otherwise exposed to radiation. In other cases, treating radiation injury can include mitigating chemical toxicity. For example, treating radiation injury can comprise mitigating an effect of chemical toxicity (e.g., hematologic toxicity, gastrointestinal toxicity) in a subject receiving radiation therapy or otherwise exposed to radiation.

As used herein, the terms “effective amount” or “therapeutically effective amount” refer to a sufficient amount of a compound being administered which will relieve to some extent one or more of the symptoms of a radiation injury for which the subject is being treated. The result can be reduction and/or alleviation of the signs, symptoms, or causes of a radiation injury, or any other desired alteration of a biological system. For example, an “effective amount” for a method provided herein can be the amount of a compound described herein (e.g., an APC or PC polypeptide) that is required to provide a clinically significant decrease in any aspect of a radiation or chemical injury. An effective amount can vary depending on, inter alia, the APC or PC polypeptide administered to the subject, the type of radiation injury and its severity, and the age, weight, etc., of the subject to be treated. An appropriate effective amount in any individual case may be determined using techniques known to those in the art, such as a dose escalation study.

In another aspect, the present invention provides methods for preventing radiation injury in a subject. As used herein, the terms “preventing” and “to prevent” refer to proactively limiting, diminishing, mitigating, or lessening the severity of any aspect of radiation injury in a subject. In an exemplary embodiment, preventing radiation injury includes taking proactive or prophylactic measures to eliminate or reduce the risk or severity of radiation injury in the subject upon radiation exposure. In some cases, a method for preventing radiation injury in a subject comprises administering to the subject an effective amount of APC, PC, or a variant thereof as described herein. In some cases, the effective amount is administered to the subject at one or more time points prior to or during radiation exposure.

In yet another aspect, the present invention provides methods for reducing chemical toxicity in a subject receiving myelosuppressive therapy. As used herein, the term “myelosuppressive therapy” refers to any therapy or treatment regimen which includes administering one or more agent that slows or inhibits normal blood cell and platelet production in the bone marrow. Many chemotherapeutic agents are myelosuppressive agents. For example, chemotherapeutic agents such as cisplatin, carboplatin, 5-fluorouracil, bleomyocin, spiroplatin, marcellomycin, mitomycin C, doxorubicin, etoposide, cyclophosphamide, and bis-chloroethylnitrosourea (BCNU) exert myelosuppressive effects on bone marrow. These chemotherapeutic agents are also myeloablative agents. As used herein, the terms “myeloablative” and “myeloablation” refer to severe or complete depletion of bone marrow cells resulting from, in some cases, administration of high doses of chemotherapy or radiation therapy. Additional myelosuppressive and myeloablative agents are known and available to those who practice in the art.

As used herein, the term “reducing chemical toxicity” refers to eliminating, mitigating, or lessening the severity of any aspect of the toxicity of chemical compounds towards normal cells and tissues (e.g., hematologic toxicity, gastrointestinal toxicity) including, for example, toxicity resulting from myelosuppressive therapy or another form of chemical toxicity. Chemical toxicity could also result from exposure of the subject to enterotoxin, phytotoxin, heavy metals or other chemicals known to damage the hematopoeitic or gastrointestinal systems. In some cases, chemical toxicity is assessed by detecting apoptosis in normal tissues, detecting blood toxicity by monitoring hematological syndromes (e.g., neutropenia, anemia, and thrombocytopenia), detecting atypical bleeding (e.g., gastrointestinal bleeding), or detecting any genotoxic effects of a myelosuppressive therapy.

In some cases, a method for reducing chemical toxicity in a subject receiving myelosuppressive therapy comprises administering to the subject an effective amount of an APC or PC polypeptide as described herein.

Scenarios of Treatment:

In addition to the uses described herein for an APC or PC polypeptide or variant, therapeutic or preventive administration of APC or PC polypeptide can be beneficial for at least the following scenarios: (1) post-exposure treatment of an individual accidentally exposed to disease-causing doses of ionizing radiation or chemical toxicity; (2) treatment before, during, or after exposure of individuals involved in decontamination or first-responder efforts related to accidents involving the release of radioactive or chemical material; (3) treatment before, during, or after exposure of individuals undergoing partial or whole body irradiation during the course of anticancer therapy; (4) treatment before, during or after exposure of individuals undergoing targeted irradiation or chemotherapy during the course of anti-cancer therapy; (5) preventive or post-exposure treatment of individuals or animals exposed to radiation from environmental sources, as it may occur in space flight, mining operations, or radon-rich areas; (6) treatment of valuable livestock animals exposed to disease-causing doses of ionizing radiation or chemical toxicity as in myeloablation or accidental release.

Formulation:

Pharmacologic formulations, single or multiple amino-acid substitutions or additions or deletions, post-translational modifications, or other treatments (which may collectively be referred to as “alterations”), or any combination of such “alterations” may favorably or unfavorably alter the bioactivity and/or pharmacokinetic properties, resulting in altered efficacy, uptake, biodistribution, half-life, altered rate of conversion into APC, resistance to processing or inhibition by natural endogenous molecules, interaction with other medications or non-natural modifiers of efficacy, depot effects, or clearance upon i.v. delivery or upon delivery via other routes, such as oral ingestion, subcutaneous injection, transdermal diffusion, rectal introduction, intraperitoneal injections, inhalations or delivery through other anatomical routes. Dosing will be adjusted by a skilled person knowledgeable in the state-of-the-art of evaluating the pharmacokinetic properties of the drug to produce a dose range that produces efficacy and/or circulating levels of APC or PC polypeptide that is comparable to that achieved by intravenous infusion (bolus or continuous) of normal APC or PC zymogen.

In some cases, APC, PC, or a variant thereof is provided in a pharmaceutical formulation for convenient administration of a dose to the subject. For example, a pharmaceutical formulation for use according to the methods provided herein can comprise a polypeptide and a pharmaceutically acceptable carrier, diluent, or excipient. As used herein, the term “pharmaceutically acceptable carrier, diluent, and excipient” refers to vehicles or additives conventionally used in formulating pharmaceutical compositions. Vehicles and additives can include any and all excipients, solvents, dispersion media, coatings, antibacterial and antifungal agents, toxicity agents, buffering agents, absorption delaying or enhancing agents, surfactants, and miclle forming agents, lipids, liposomes, and liquid complex forming agents, stabilizing agents, and the like. Pharmaceutically acceptable carriers appropriate for methods of the present invention can include at least one excipient such as sterile water, sodium phosphate, mannitol, sorbitol, or sodium chloride, or any combination thereof. Other pharmaceutically acceptable carriers which can be used include, without limitation, solvents or dispersion medium containing, for example, water, ethanol, polyol (e.g., glycerol, propylene glycol, liquid polyethylene glycol), suitable mixtures thereof, and vegetable oils. The use of such media and agents for pharmaceutically active substances is known in the art. In some cases, supplementary active compounds are also incorporated into the pharmaceutical formulations.

Administration:

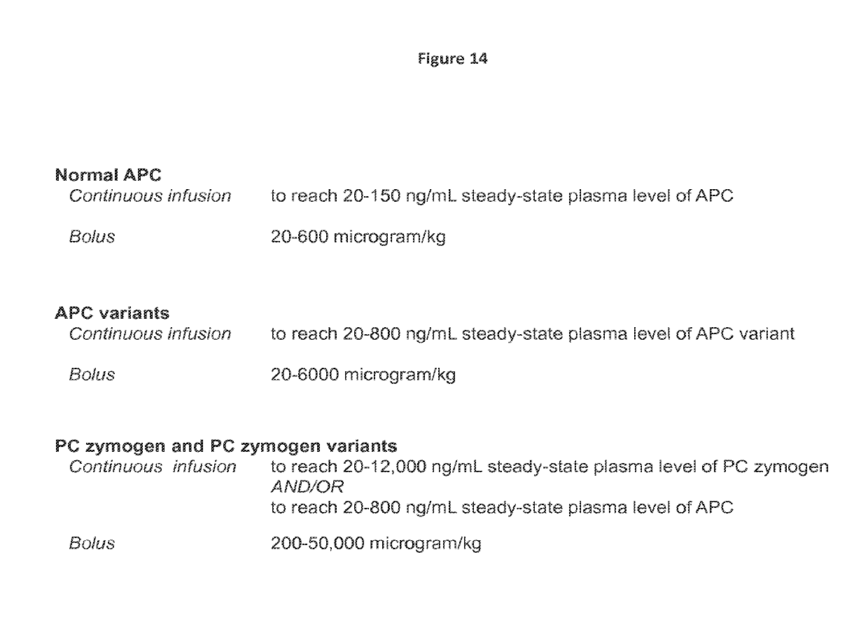

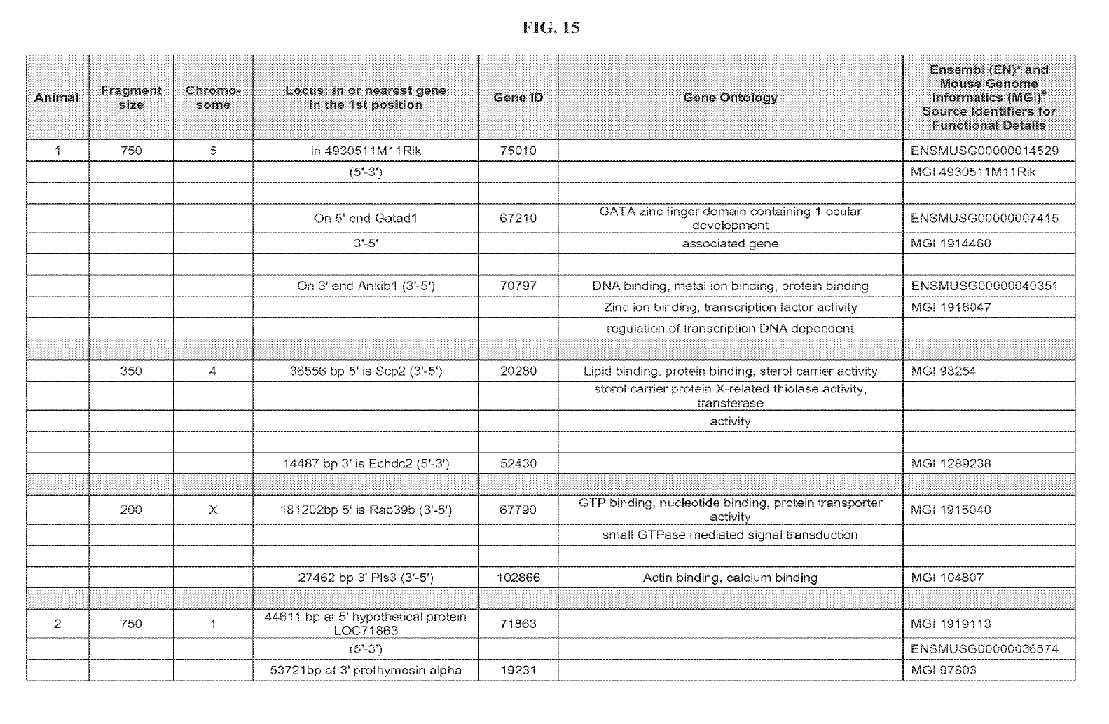

For the methods provided herein, APC, PC, or a variant polypeptide, or a pharmaceutical composition comprising APC, PC, or a variant polypeptide can be administered by any means that achieves the intended purpose or is deemed appropriate by those of skill in the art. For example, the polypeptide can be administered by intravenous infusion, intraperitoneal injection, or another mode. Infusion can be a continuous intravenous drip infusion, a single intravenous bolus infusion, multiple intravenous bolus infusions which are, in some cases, separated by some intervening period of time, or a combination thereof. In an exemplary embodiment, administering APC or PC polypeptide can comprise a continuous intravenous infusion beginning prior to exposure or as early as possible after radiation exposure and, preferably, within 24 hours (e.g., within about 1 minute, 5 minutes, 30 minutes, 1 hour, 2 hours, 3 hours, 5 hours, 8 hours, 12 hours, 15 hours, 18 hours, 20 hours, 24 hours) of the radiation exposure according to the doses outlined in FIG. 14. In another exemplary embodiment, administering APC, PC, or a variant polypeptide comprises a single or repeated bolus infusion according to the dose(s) outlined in FIG. 14 based on a subject's body mass. In another exemplary embodiment, administering a polypeptide provided herein comprises a repeated bolus infusion according to the dose(s) outlined in FIG. 14 with bolus doses spread out over a treatment period of one to at least several days. In another embodiment, bolus doses would occur one to six times each 24 hour period. In some cases, repeated infusions of the bolus are spaced at intervals of about 1 to about 24 hours. In some cases, it may be necessary to delay the start of treatment of the subject with an APC or PC polypeptide for up to three days following exposure to the radioactive or chemical toxicity. In another exemplary embodiment, administering an APC or PC polypeptide provided herein can comprise a single or repeated intraperitoneal injection of a bolus of APC or PC polypeptide, where the intraperitoneal dose is 10- to 20-fold higher than a dose used for an intravenous infusion.

Dosing:

For molecules that are infused intravenously, but readily diffuse out of the bloodstream into tissues, the body surface area may be used as one guide to convert drug doses between different species and adjust dosing. For example, using this guide, a mouse dose of 330 microgram/kg (bolus intravenous) would be equivalent to a dose of 26 microgram/kg (bolus intravenous) in a human patient. This guide may not be fully applicable to drugs that act within the bloodstream, on cells that are in immediate contact with blood (such as endothelium or circulating cells or circulating platelets, or other blood components), or on an anatomical locale in close proximity to a blood-containing space. In the case of the drug acting within the bloodstream or endothelial cells lining a blood vessel, a mouse steady-state plasma level of the drug of 50 ng/mL would be approximately equivalent to a steady state plasma level of 50 ng/mL in a human patient. Dosing predictions can be extrapolated from murine dosages, where a dosage of 10 μg/mouse corresponds to a human dose of approximately 330 μg/kg. Single or multiple administrations of 330 μg/kg can be used for a human patient. Determining an appropriate dosage range should consider the half-life of the polypeptide to be administered. For example, the half-life of APC or recombinant human wild-type APC following administration to a mouse is approximately 12 minutes, while its half-life following administration to a human is approximately 15-20 minutes.

Expression of the radiation-protective bioactivity of PC Zymogen requires unmasking of the reactive center of the protease domain of PC. This is physiologically achieved by proteolytic processing of PC zymogen by thrombin or, more efficiently, by the thrombomodulin-thrombin complex to remove a small “activation peptide” from the PC zymogen. Alterations to PC zymogen that affect this activation step in vivo through proteolysis or other mechanisms would affect dosing. This class of “activation variants” can be envisioned to be combined with any other modification in APC or in PC zymogen that would favorably alter the overall efficacy of the variant in the invented application for mitigation of radiation injury.

The precise dosing, dosing regimen, and specific choice of APC or PC polypeptide used according to the methods provided herein may vary depending on the specific circumstances of radiation exposure or imminent radiation exposure. Such circumstances can include, without limitation, availability of resources for various routes of administration and access to subjects in need of treatment according to a method provided herein.

Bioactivity Considerations:

The invention is based on the discovery that wild-type APC polypeptide exhibits a bioactivity that mitigates radiation toxicity. Although specific APC and PC variants are described herein, the present invention relates to polypeptides that mitigate radiation toxicity—polypeptides that need not comprise an amino acid sequence variation and need notexert this activity to a greater extent relative to wild-type APC or exhibit this bioactivity without exerting potentially undesirable effects. Bioactivity that mitigates radiation toxicity may involve one or more known or unknown biochemical functions of APC. Some functions of APC are known to have undesirable side effects that are observed once a given level of APC is exceeded. For example, the anticoagulant effect of APC may set an upper limit on an acceptable dose. Because pharmacologic formulation, amino-acid substitutions or additions or deletions or combinations, post-translational modifications, or other treatments, may affect the various bioactivities and biochemical functions of APC, either selectively or non-selectively, any specific variant may be effective within the same dose range, a partially overlapping dose range, or a non-overlapping dose range as normal APC.

Other Cases:

In one example, an APC or PC polypeptide with 5 to 10-fold reduced in vivo anticoagulant activity, but normal radio-protective activity could be administered at 5-to-10-fold higher doses without reproducing the undesirable side-effects attributed to APC's anticoagulant activity.

In another example, an APC or PC polypeptide with 5 to 10-fold (on a molar basis) increased radioprotective activity against pathologies resulting from radiation exposure, but normal other APC functions, could be administered at 5 to 10-fold lower doses than normal APC.

In some cases, the methods provided herein further comprise administering additional agents to a subject. For review, see Berbée & Hauer-Jensen, Curr. Opin. Support Palliat. Care 6(1):54-59 (2012). For example, a method can further comprise administering an anti-oxidant to a subject. An anti-oxidant appropriate for the methods provided herein can be the vitamin E analogue, γ-tocotrienol. In some cases, a method can further comprise administering a Toll-like Receptor 5 (TLR5) agonist to a subject. A TLR5 agonist appropriate for the methods provided herein can be the synthetic flagellin derivate, CBLB502.

For the methods provided herein, the subject can be exposed to radiation of any type and from any appropriate source. For example, a subject can be exposed to ionizing radiation. Ionizing radiation produces chemically reactive ions that cause significant biological damage per unit of energy of ionizing radiation. Sources of ionizing radiation include, without limitation, cosmic radiation, nuclear medicine, x-rays, nuclear fuel, and nuclear fallout. In some cases, the radiation is non-ionizing. Non-ionizing radiation does not produce charged ions, but has sufficient energy to excite electrons to higher energy states.

### Embodiments

In a preferred commercial embodiment, the subject invention is a method to treat a subject exposed to a lethal or sub-lethal dose of radiation with an APC or PC polypeptide by administering to said subject an intravenous or intraperitoneal infusion of said polypeptide in an effective dose to mitigate radiation toxicity.

In another embodiment, the subject invention is a method to treat a subject exposed to a lethal or sub-lethal dose of chemicals used to myeloablate a subject with APC or PC polypeptide by administering to said subject an intravenous or intraperitoneal infusion in an effective dose to mitigate chemical toxicity.

In another embodiment, the subject invention is a method to prevent toxicity to radiation or chemicals in a subject wherein an effective dose of APC, PC or a variant thereof is given either before or up to several days after exposure to the toxic insult.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

Various additional embodiments of methods according to the present invention are provided in the following non-limiting, illustrative Examples. Indeed, various modifications of the invention in addition to those shown and described herein will become apparent to those skilled in the art from the foregoing description and the following Examples and fall within the scope of the appended claims.

### EXAMPLES

### Example 1

### Infusion of Recombinant APC and APC/PC Polypeptide Variants

A series of experimental studies in laboratory mice documented that increased expression of thrombomodulin (Thbd), either by enhancing expression of endogenous Thbd or by direct overexpression of transgenic Thbd via viral transduction of hematopoietic stem and progenitor cells (HSPC), accelerated the recovery and re-expansion of the hematopoietic system after whole body exposure to non-lethal doses (3 Gy) of ionizing radiation. In contrast, reduced expression of endogenous Thbd had the opposite effect. The beneficial effect of Thbd overexpression on hematopoietic recovery from non-lethal radiation exposure was reproduced by intravenous administration of a recombinant form of soluble Thbd. Notably, therapeutic administration of Thbd within 30 minutes following radiation exposure through systemic intravenous infusion also significantly reduced overall mortality of experimental mice exposed to a lethal (LD₅₀) dose of radiation.

Since one of several biological activities of Thbd is the activation of the zymogen protein C into the active serine protease form APC, assays were performed to determine the radioprotective effect of APC. A single intravenous infusion of a bolus of recombinant murine APC (0.3 mg per kg body weight), given within 30 minutes after exposure to a lethal dose of radiation, significantly improved 30-day survival. Likewise, APC treatment also accelerated hematopoietic recovery. These data demonstrated that APC is a critical, and likely sufficient, downstream effector that mediates the radio-protective effects of Thbd. Multiple bolus infusions of APC also significantly reduced mortality after exposure to a single dose of radiation (10.5 Gy) that causes near complete mortality in untreated mice. The therapeutic efficacy of APC in these preclinical animal models, therefore, approaches a remarkable dose-reduction-factor of approximately 1.2. The therapeutic window of opportunity for successful treatment with APC (i.e., mortality reduction) extends to 24 hours or more after exposure to lethal doses of whole body radiation exposure.

A comparable therapeutic efficacy is obtained with E149A-APC, a recombinant variant of APC comprising a single amino acid residue substitution of the glutamic acid at position 149 for an alanine residue. The biological activity of APC variants such as E149A-APC is preserved but a smaller quantity of a variant may be necessary to achieve a level of protection from radiation injury that is comparable to that of wild-type APC. In some cases, an APC variant may be superior to normal APC in at least two respects: (1) requiring a lower dose for achieving the same level of efficacy as normal APC and (2) minimizing potentially undesirable effects elicited by wild-type APC. One skilled in the art is able to determine the potential superiority of APC, PC or variants thereof for radioprotection through the use of appropriate experiments such as a dose escalation study.

It was further determined that continuous low-dose (approximately 50-100 ng/mL at steady state) in vivo production of a hyperactivatable human Plasma Zymogen Protein C (“hyperactivatable” PC), which is achieved by constitutive transgenic expression of the zymogen protein in the liver, afforded a mortality reduction after a LD₅₀ of whole body radiation exposure that was equivalent to that obtained by bolus infusion of APC. Hyperactivatable Plasma Zymogen Protein C contains 2 amino acid substitutions spanning the activation peptide cleavage site (D167F/D172K), has an increased half-life relative to APC, and is efficiently activated by thrombin alone or by the Thbd-thrombin complex. See Richardson et al., Nature 360:261-264 (1992); and Isermann et al., Nat. Med. 13:1349-58 (2007). The steady state levels of APC achieved in these mice closely resemble the levels achieved by the clinical dosing-regimen of human recombinant APC in the treatment of sepsis, which is achieved with a dose sufficient to yield a steady-state plasma level of approximately 50 ng/mL APC.

Studies with these hyperanticoagulant APC or hyperactivatable PC variants demonstrated that the therapeutic efficacy of APC is maintained with different modes of administration or dosing and, therefore, should be adaptable to various therapeutic regimens adapted to specific circumstances. Of note, no increased mortality, increased morbidity of survivors, or spontaneous GI bleeding was ever observed in these experiments, suggesting that APC therapy is well tolerated.

The radioprotective effect of the E149A variant may be due to a mechanism independent of APC's anti-thrombotic properties. The biological mechanism underlying the radioprotective effects of APC variants may be attributed to two related pathways, i.e., enhanced survival and/or function of endothelial cells in bone marrow blood vessels that constitute part of the anatomical bone marrow niche necessary for recovery of hematopoiesis and the accelerated expansion of hematopoietic progenitors. The mechanisms engaged by APC are distinct from the principle of action of other emerging treatments for radiation-induced injury, i.e., the neutralization of reactive oxygen species and the activation of toll-like receptor 5 (TLR5), suggesting that therapy with APC will produce additive or synergistic benefits with therapies targeting reactive oxygen species or TLR5.

### Example 2

### Screening for Novel Genes and Pathways Involved in Radiation Mitigation

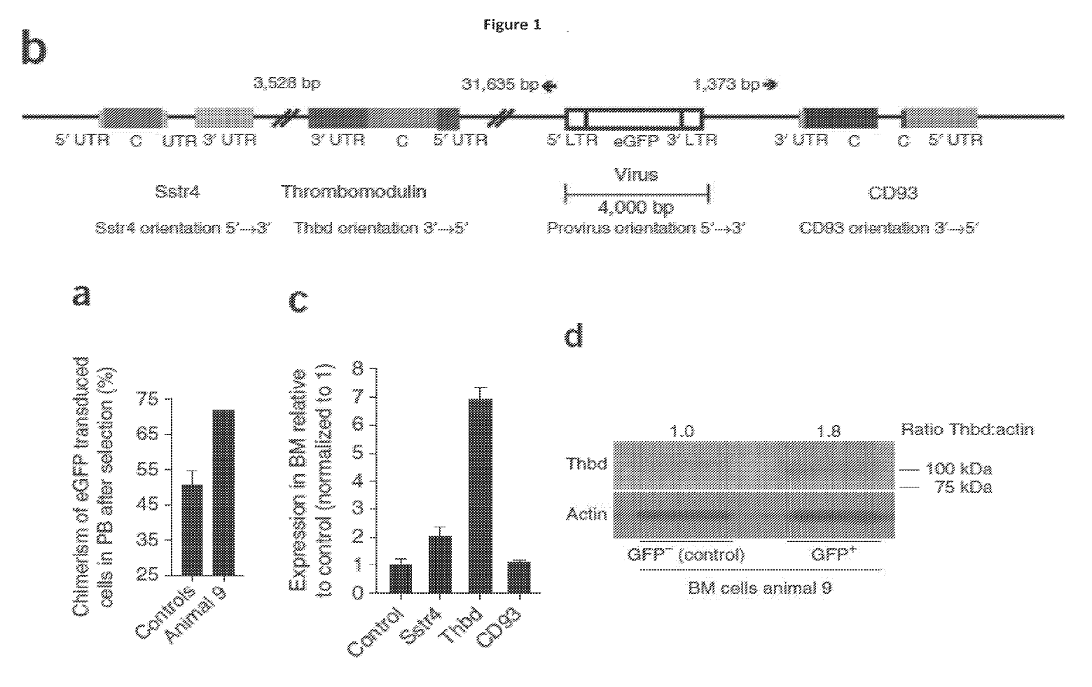

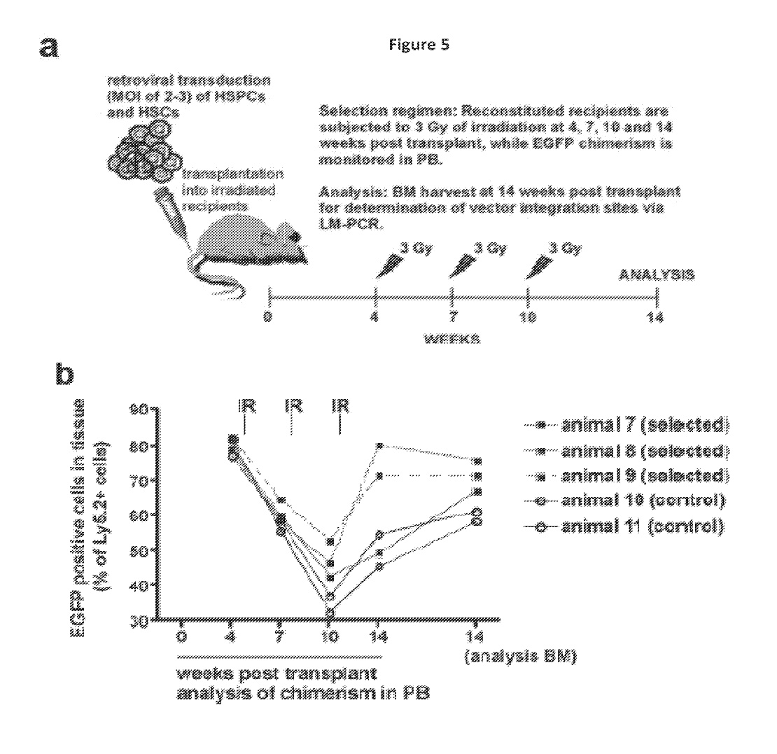

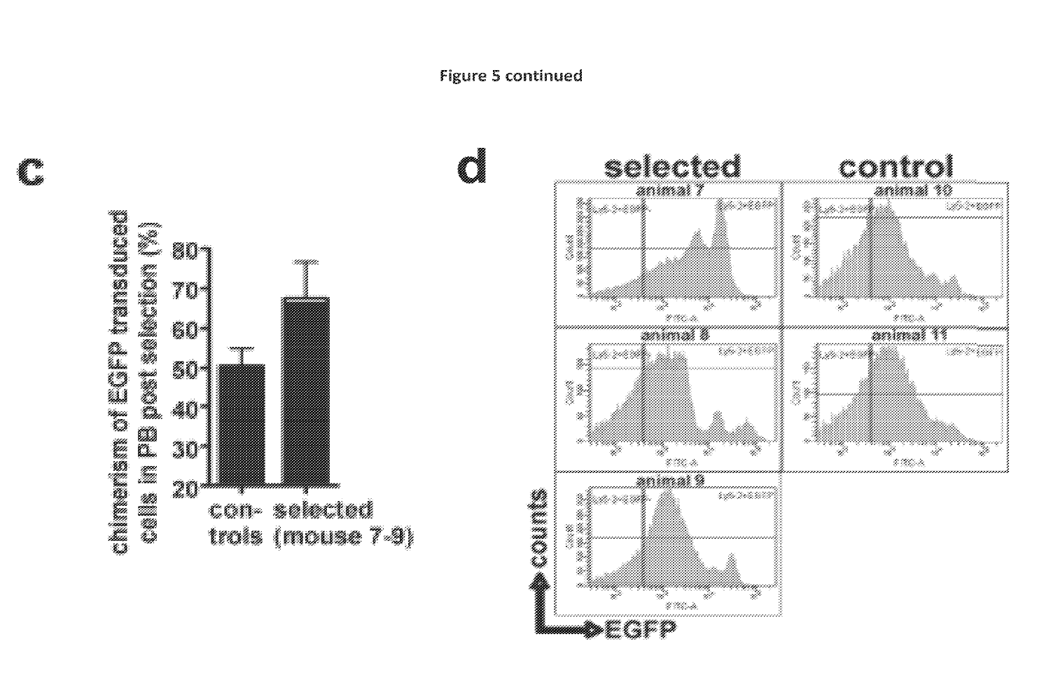

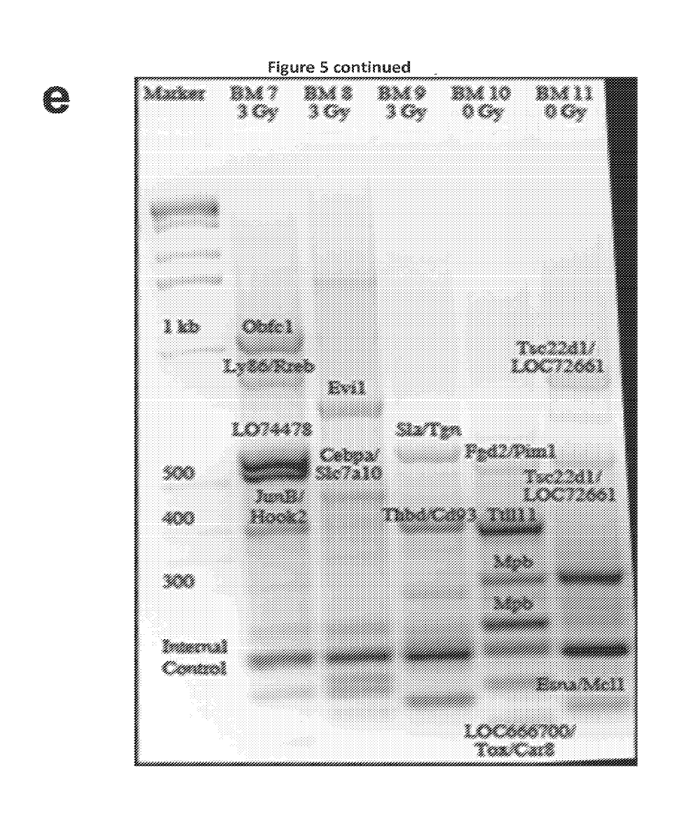

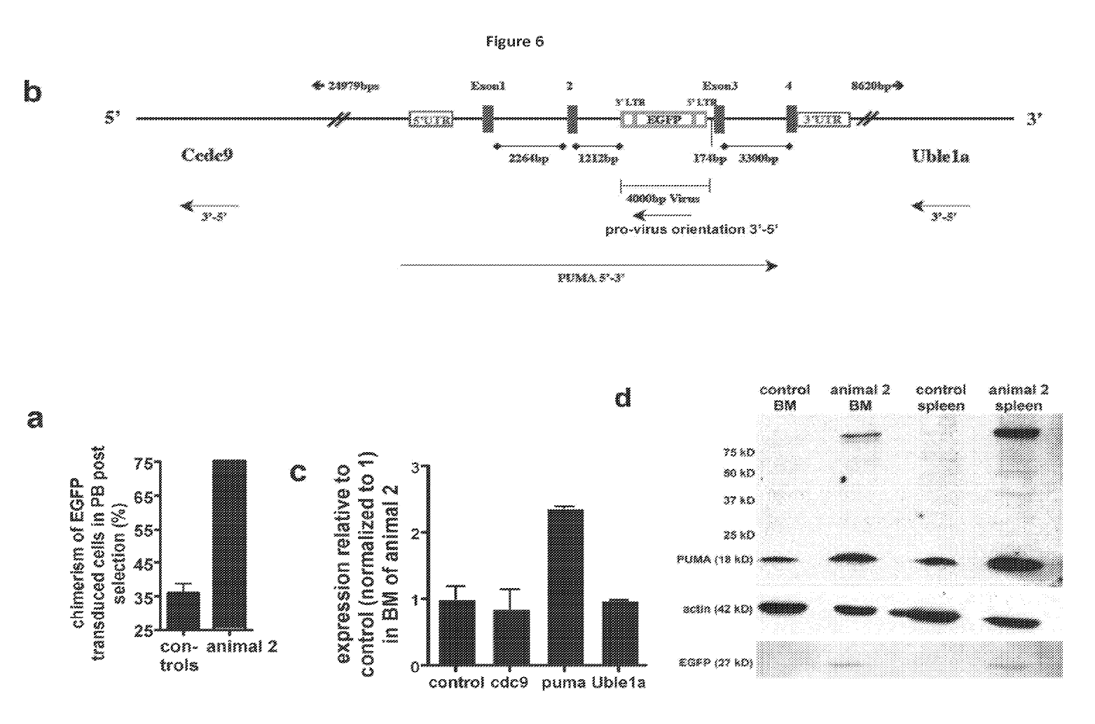

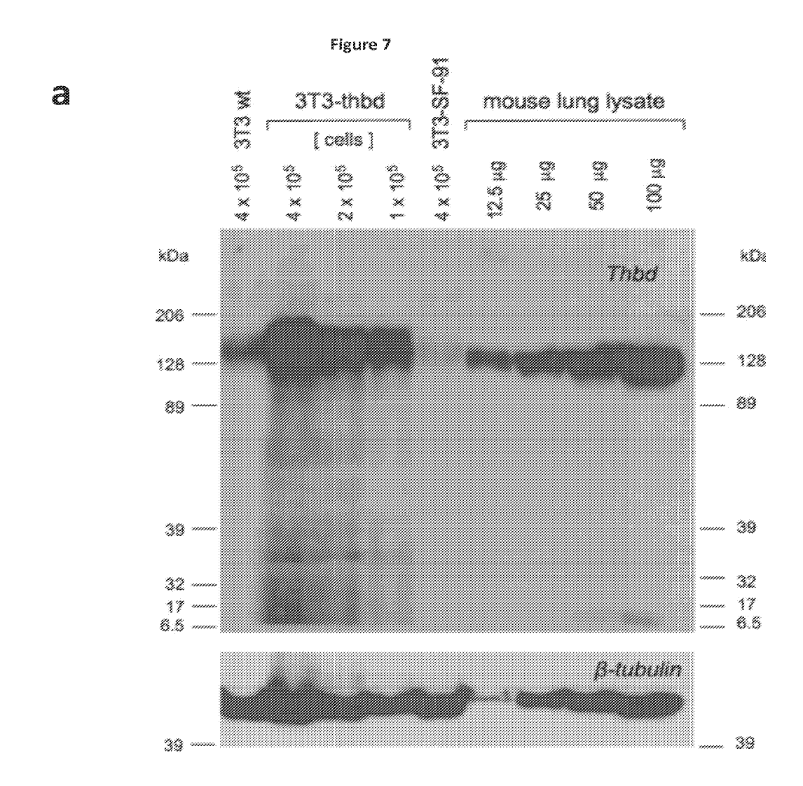

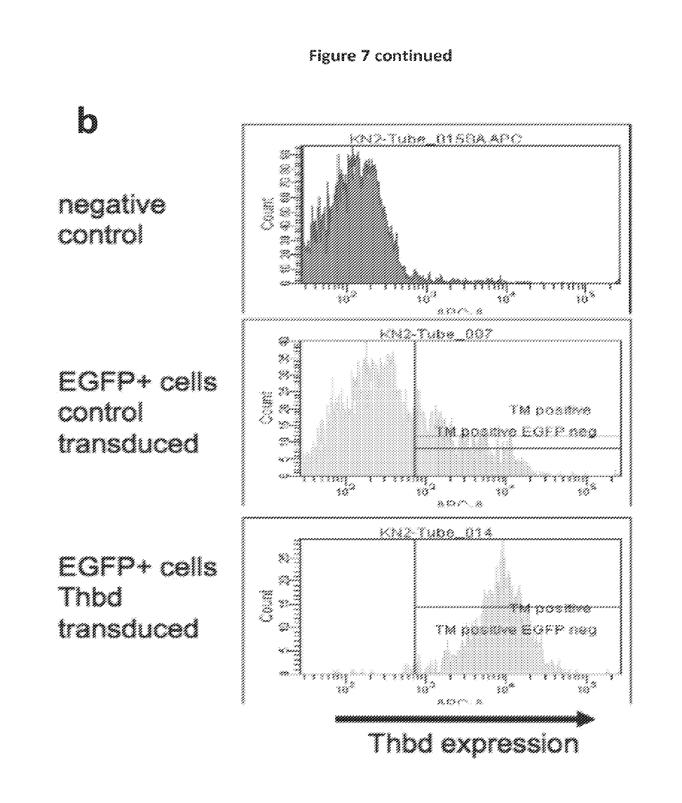

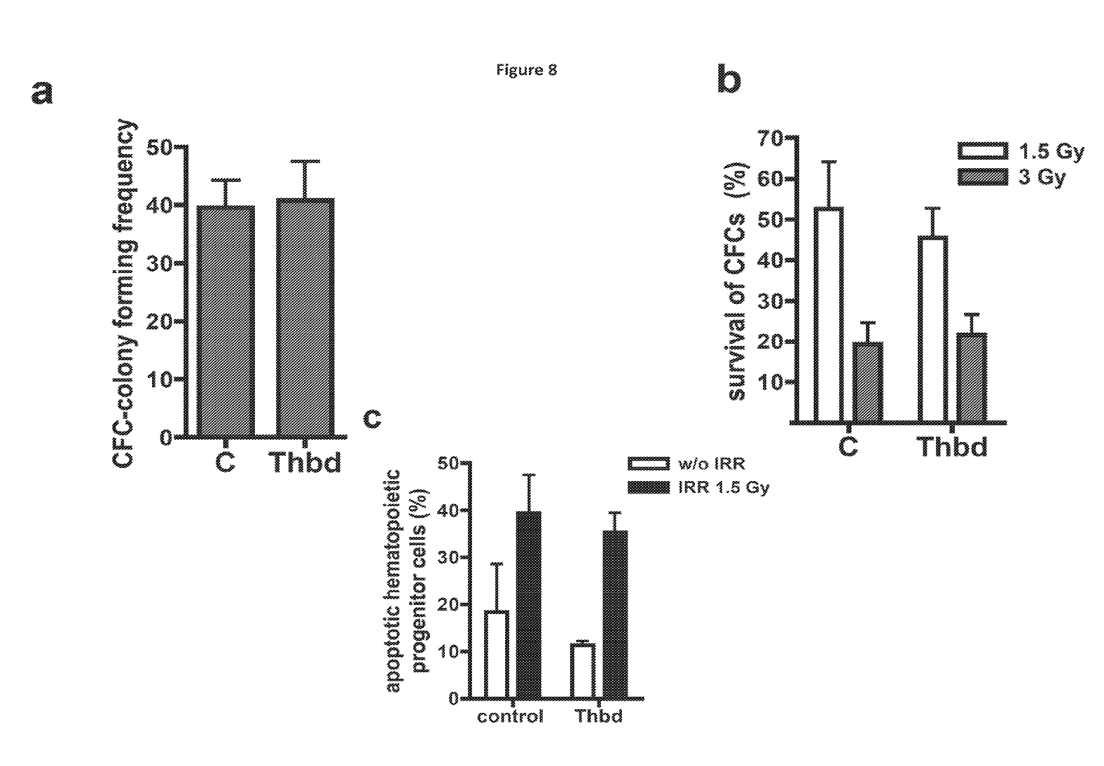

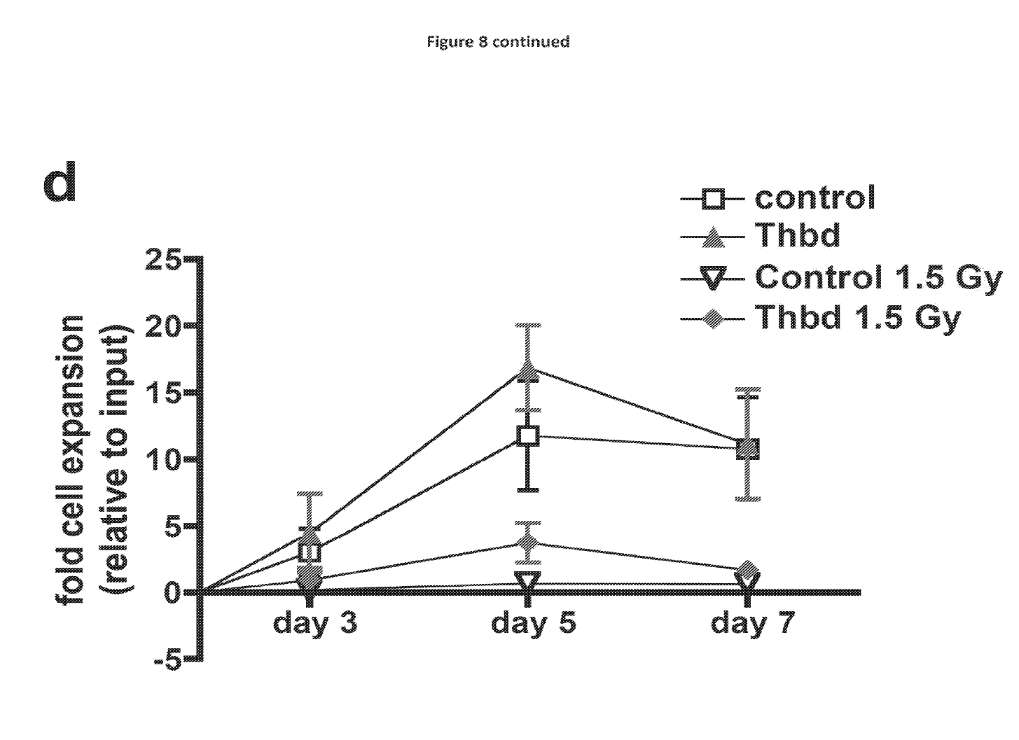

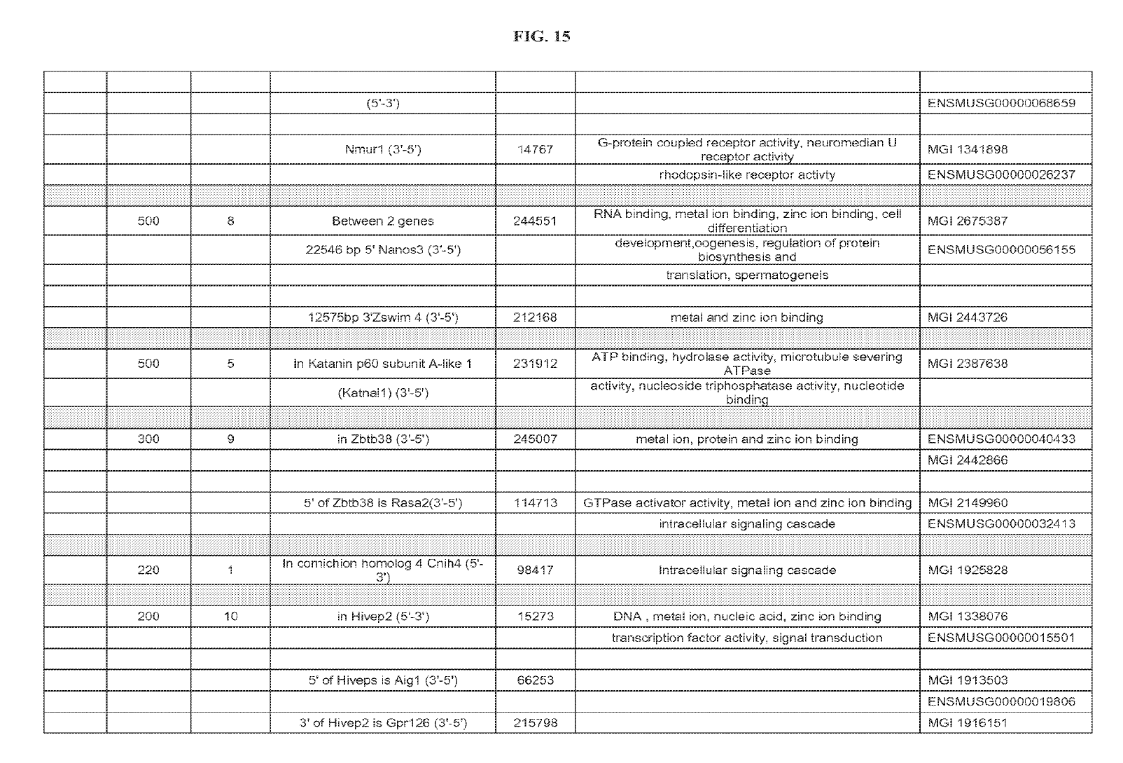

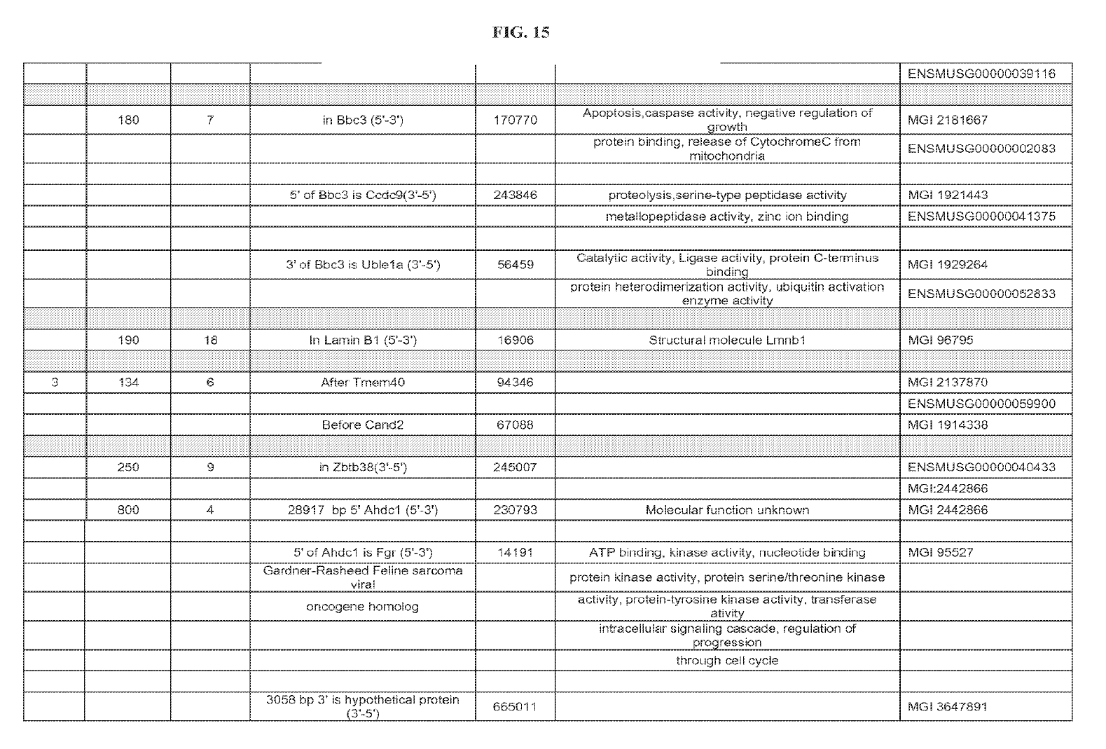

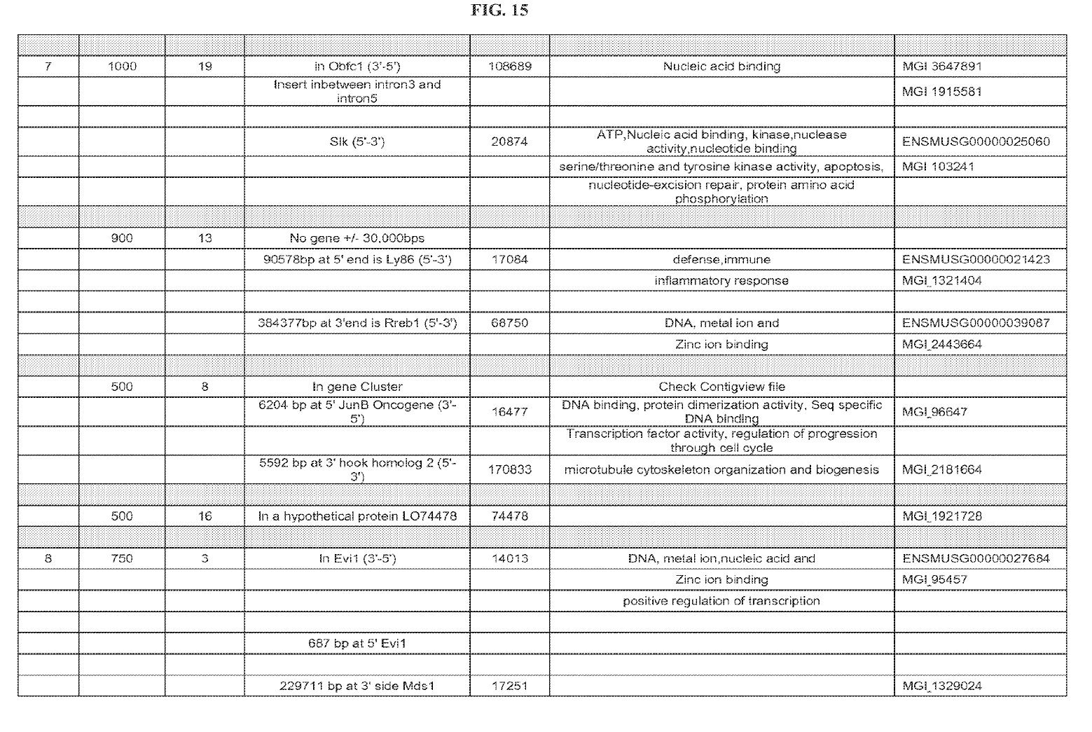

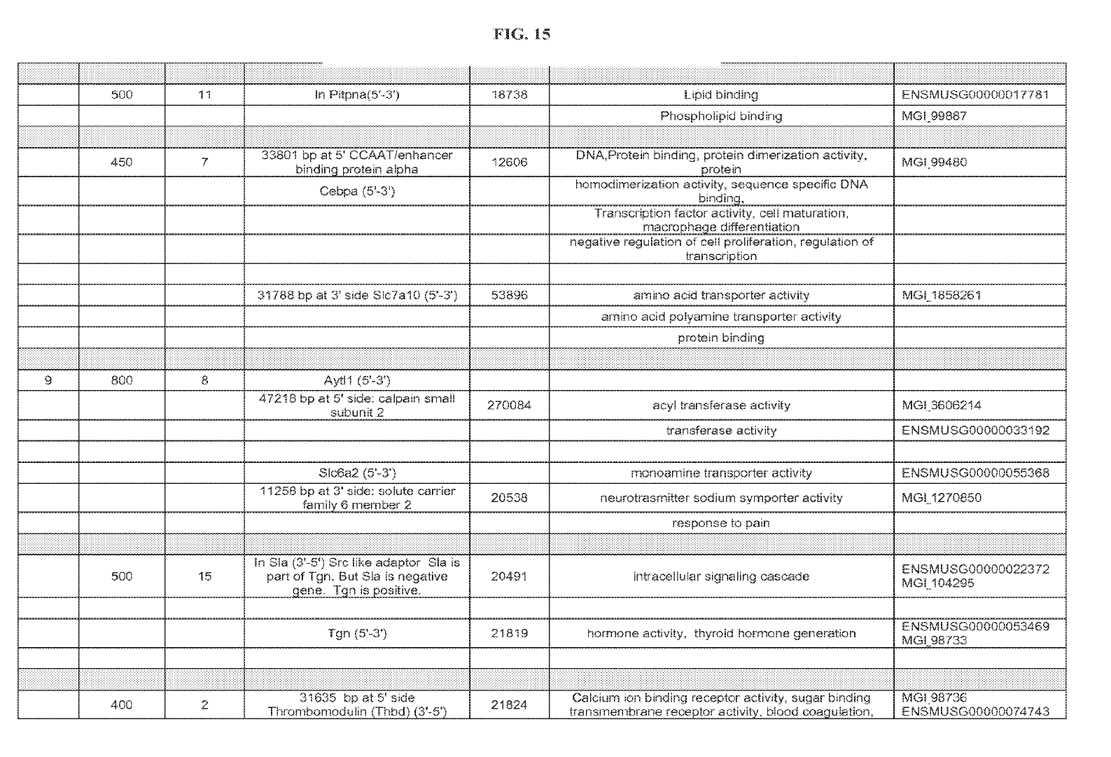

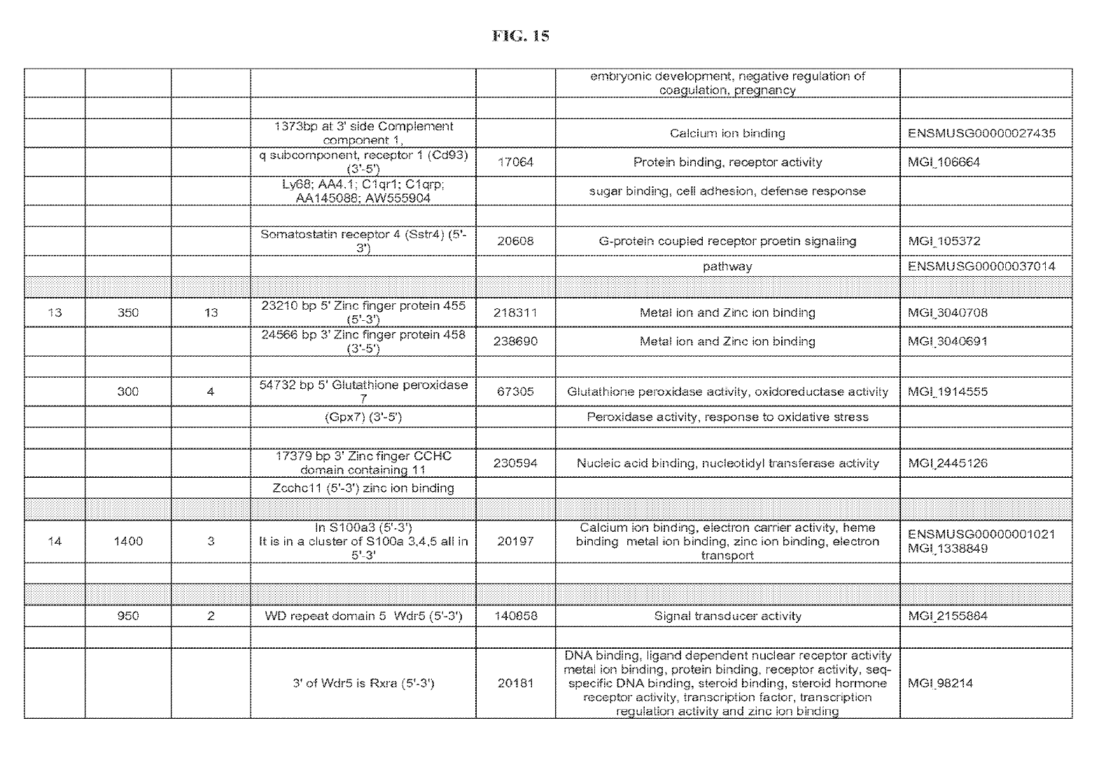

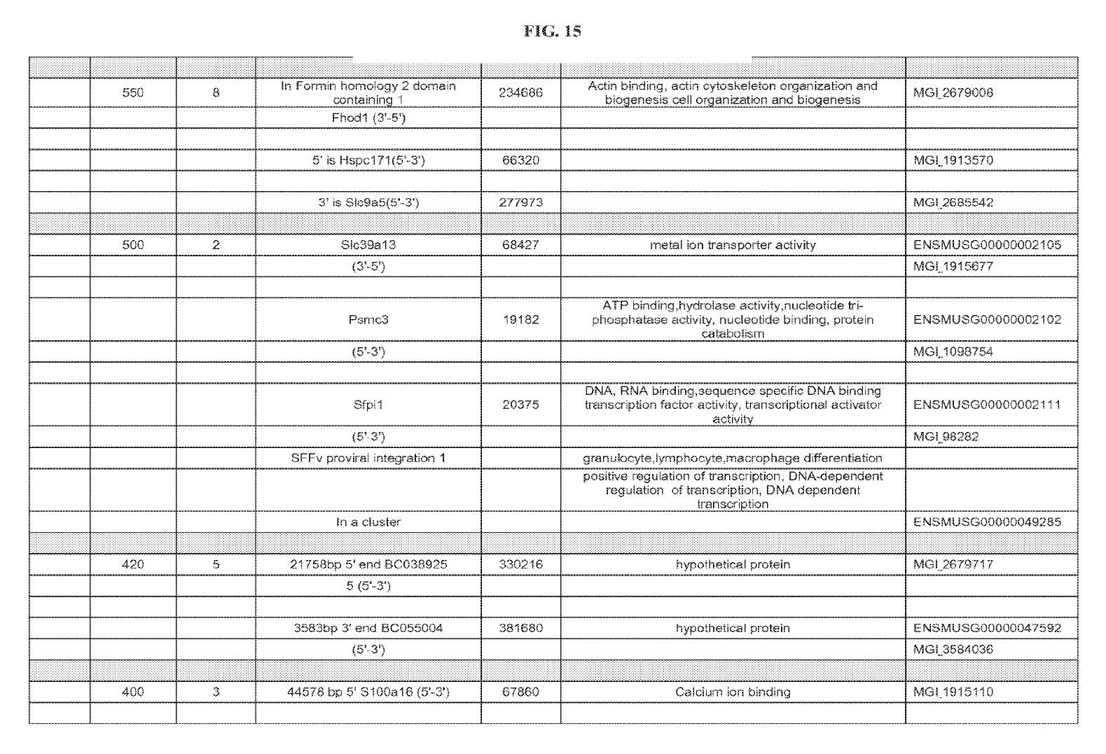

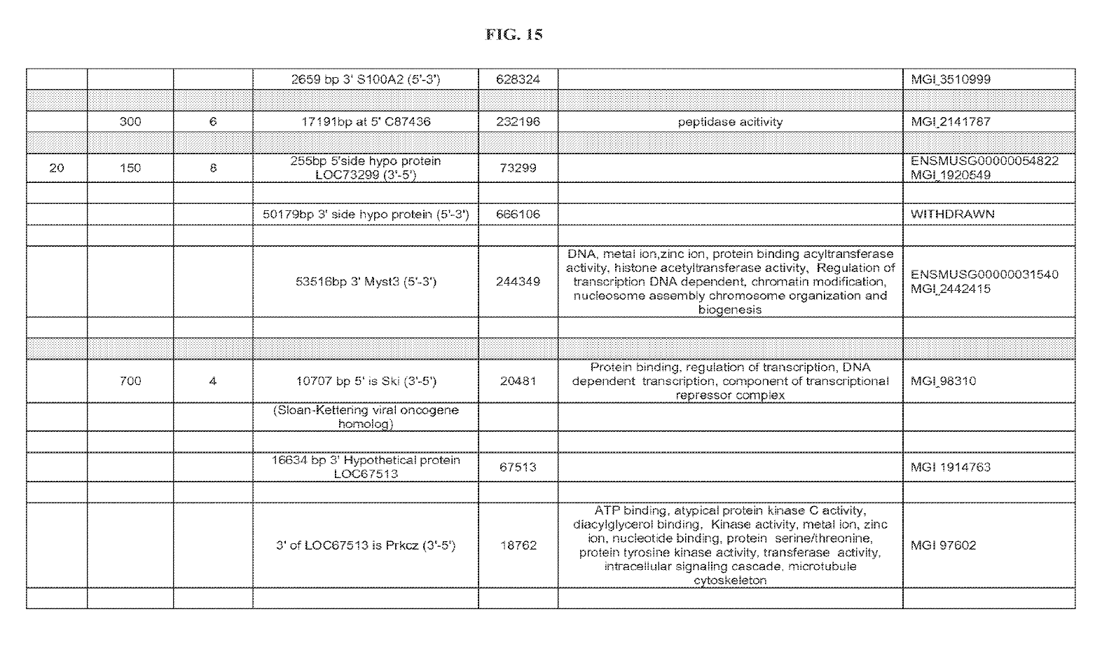

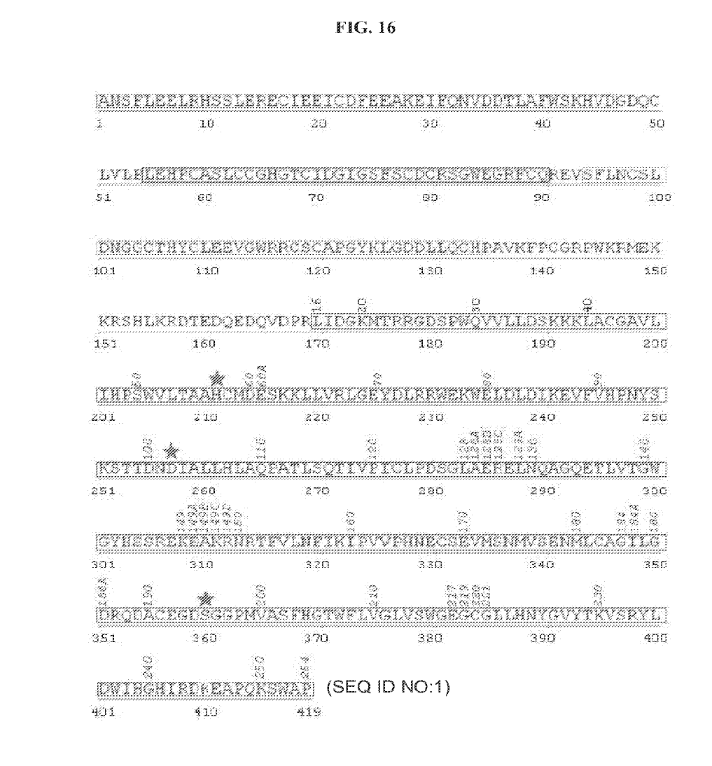

To identify novel genes and pathways protecting hematopoietic stem and progenitor cells (HSPCs) against radiation injury, retroviral insertional mutagenesis screens were performed using a replication deficient virus bearing a strong internal promoter expressing enhanced green fluorescent protein (EGFP) (FIG. 5A). At weeks 4, 7, and 10 following BM transfer, recipients were exposed to a single dose of 3 Gy TBI, resulting in three consecutive cycles of radiation-induced contraction and subsequent re-expansion of the hematopoietic system. Viral integration sites in genomic DNA in bone marrow cells from animals in which post-transplant TBI had resulted in a significantly augmented relative abundance of EGFP-positive cells in PB or BM were determined by ligation mediated (LM)-PCR (FIGS. 5B-E; FIG. 15). Loci targeted by integration included genes known to play a role in radioprotection of either hematopoietic or neuronal cells such as PUMA (FIGS. 6B-D) and c-Jun (data not shown). Kirsch et al., Science 327:593-596 (2010); Yu et al., Blood 115:3472-3480 (2010); Kanzawa et al., Oncogene 25:3638-3648 (2006). For Animal 9 (see FIG. 1A and FIG. 5B), LM-PCR revealed integration of the virus 31.6 kb upstream of the Thrombomodulin (Thbd) gene (FIG. 1B), which was associated with increased abundance of endogenous Thbd transcript and protein in radio-selected EGFP-positive cells, while the integration had little effect on expression of other neighboring genes like the somatostatin receptor 4 (SSTR4) or the C-type lectin transmembrane receptor CD93 (FIGS. 1C-D).

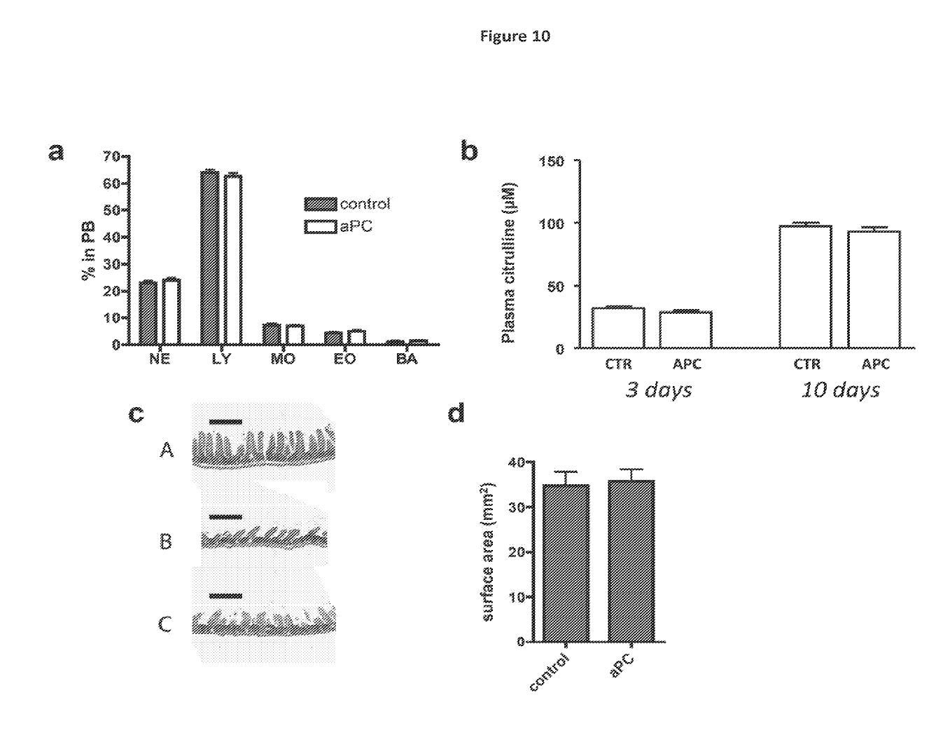

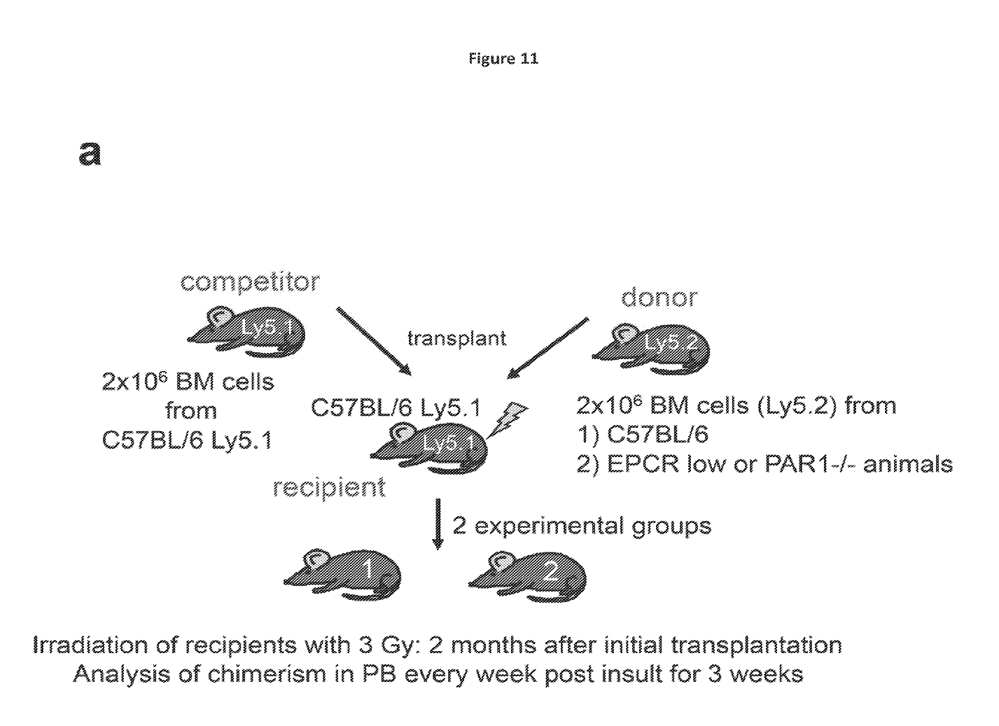

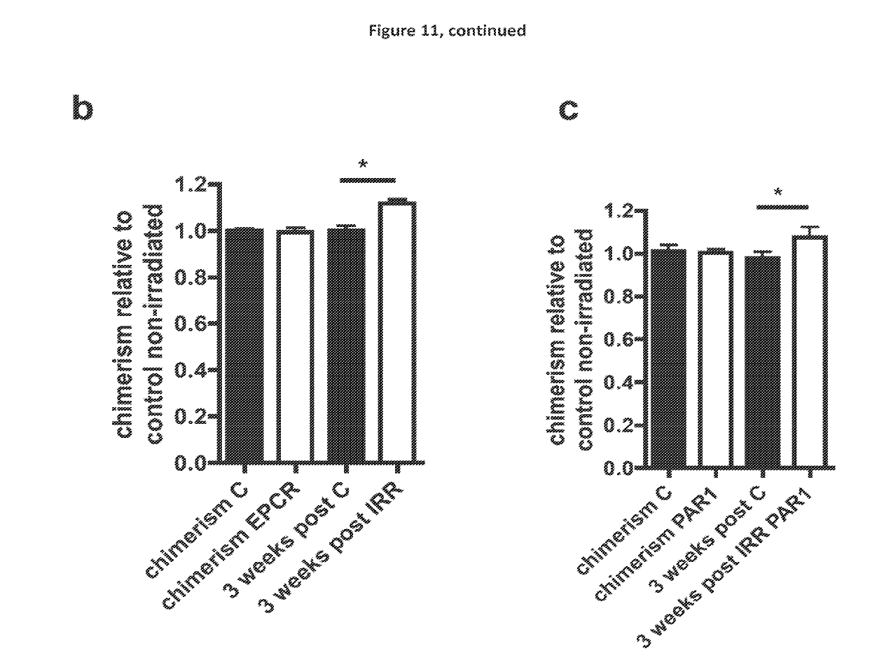

To ascertain whether augmented Thbd expression in HSPCs was sufficient for conferring a competitive selection advantage to hematopoietic cells in response to TBI, HSPCs were transduced with lentiviral Thbd-expression constructs, and Thbd over-expressing cells were subsequently transplanted into pre-conditioned C57BL/6-CD45.1 recipients (FIG. 1E and FIG. 10), followed by one 3 Gy TBI administered 4 weeks post-transplant and analysis of EGFP chimerism in PB at 3 weeks post-TBI. Cells over-expressing Thbd were 1.5-fold enriched in PB as compared to vector-only controls (FIGS. 1F-G), demonstrating that elevated expression of Thbd in hematopoietic cells was sufficient to confer a selective advantage after radiation injury. Thbd over-expressing HSPCs, however, were not protected from the effects of ionizing radiation in vitro, as determined by survival, apoptosis, and proliferation of progenitor cells in response to irradiation (FIG. 11), indicating that the beneficial effects of Thbd on HSPCs in vivo required additional cells or molecules.

Endogenous Thbd is a multifunctional cell surface-associated receptor that regulates the activities of several physiological protease systems, including complement, fibrinolysis, and blood coagulation. Weiler and Isermann, J. Thromb. Haemost. 1:1515-1524 (2003). Biochemically, Thbd functions as a high-affinity receptor for thrombin. The Thbd/thrombin complex activates thrombin activatable fibrinolysis inhibitor (TAFI) and also converts the plasma zymogen protein C (PC) into the natural anticoagulant, activated protein C (aPC). Wang et al., J. Biol. Chem. 275:22942-47 (2000); Esmon et al., J. Biol. Chem. 257:859-864 (1982); Esmon et al., J Biol Chem. 257:7944-7947 (1982). aPC inhibits blood coagulation via proteolysis of blood coagulation factors V and VIII, promotes indirectly the activity of the fibrinolytic system and exerts potent anti-inflammatory and cytoprotective effects on endothelial cells, neurons and various innate immune cell populations (Mosnier et al., Blood 109:3161-3172 (2007)) that are mediated through the interaction of aPC with signaling-competent receptors, such as Part Par2, and Par3, integrins, and the endothelial protein C receptor (Eper). Mosnier et al., supra; Weiler, Crit. Care. Med. 38:S18-25 (2010).

As the beneficial effects of Thbd in vivo could not be attributed to functions of Thbd intrinsic to HPCs, it was hypothesized that extrinsically and thus systemically administered Thbd might promote systemic beneficial effects in response to radiation injury. Administration of recombinant soluble forms of THBD to baboons and humans is safe and exhibits anticoagulant and antithrombotic activities. Tanaka et al., Br. J. Haematol. 132:197-203 (2006); van Iersel et al., Thromb Haemost. 105:302-312 (2011); Su et al., J Thromb Haemost. 9:1174-1182 (2011).

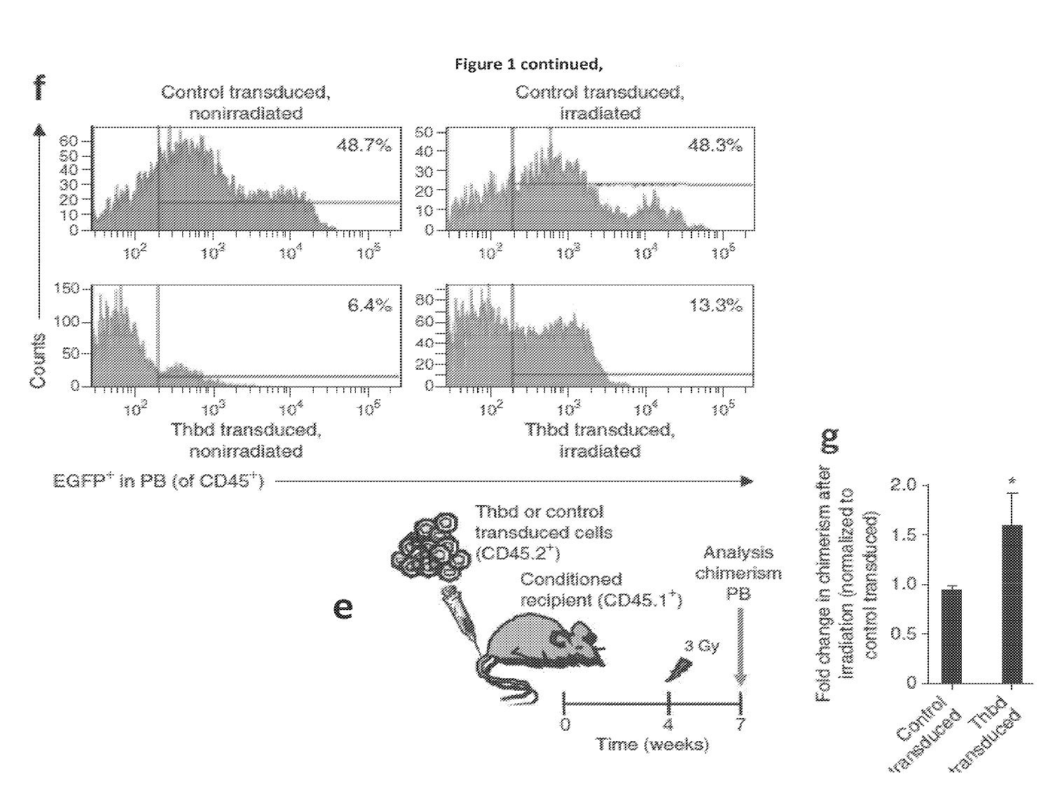

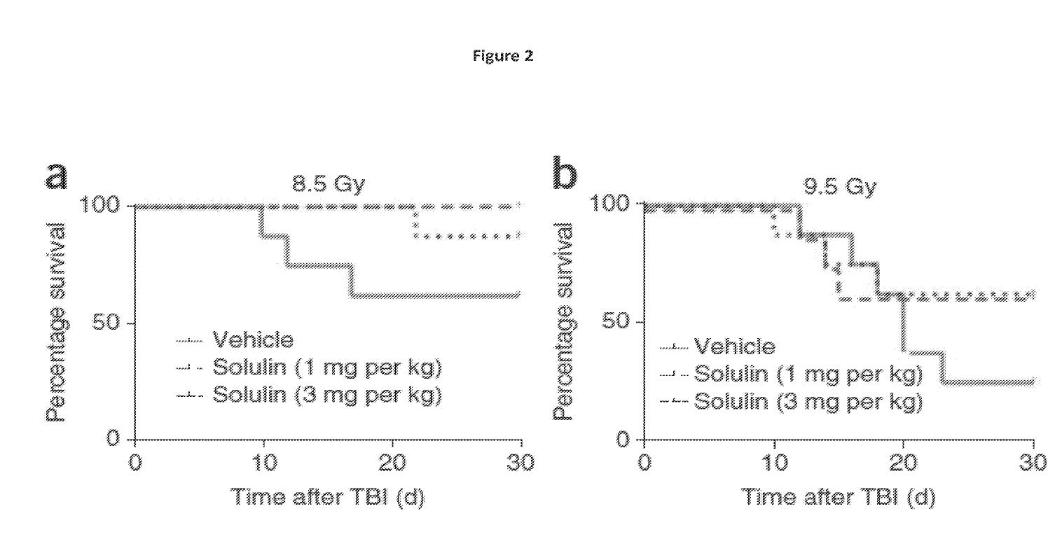

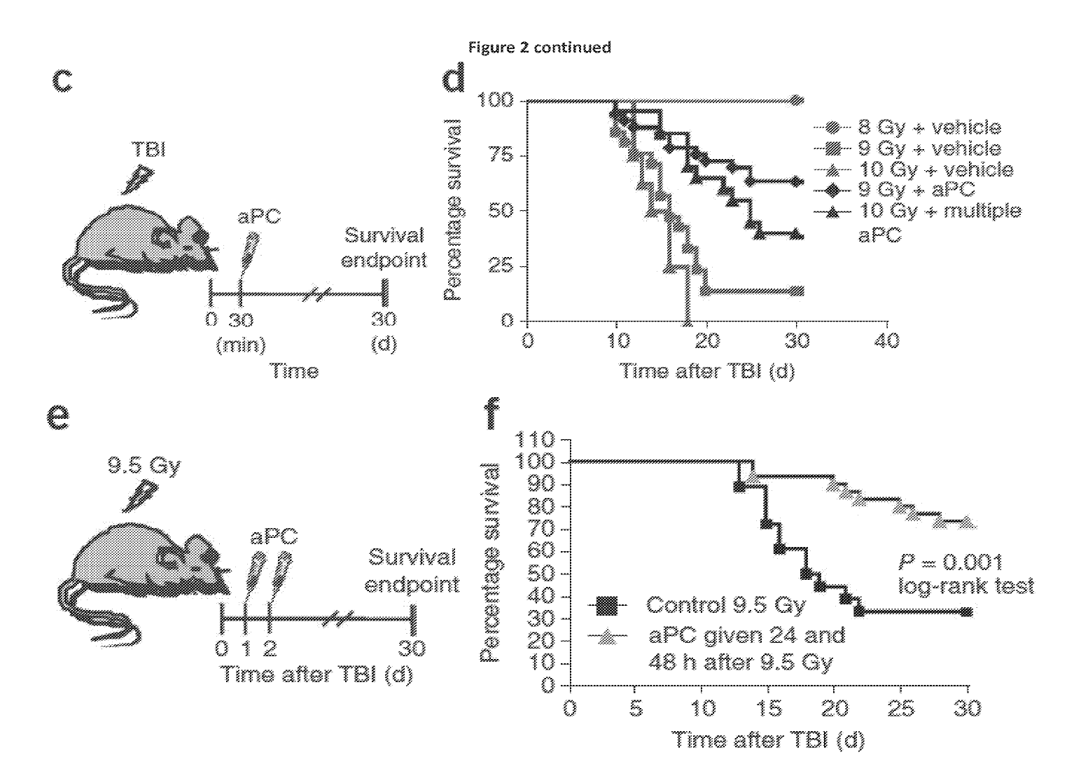

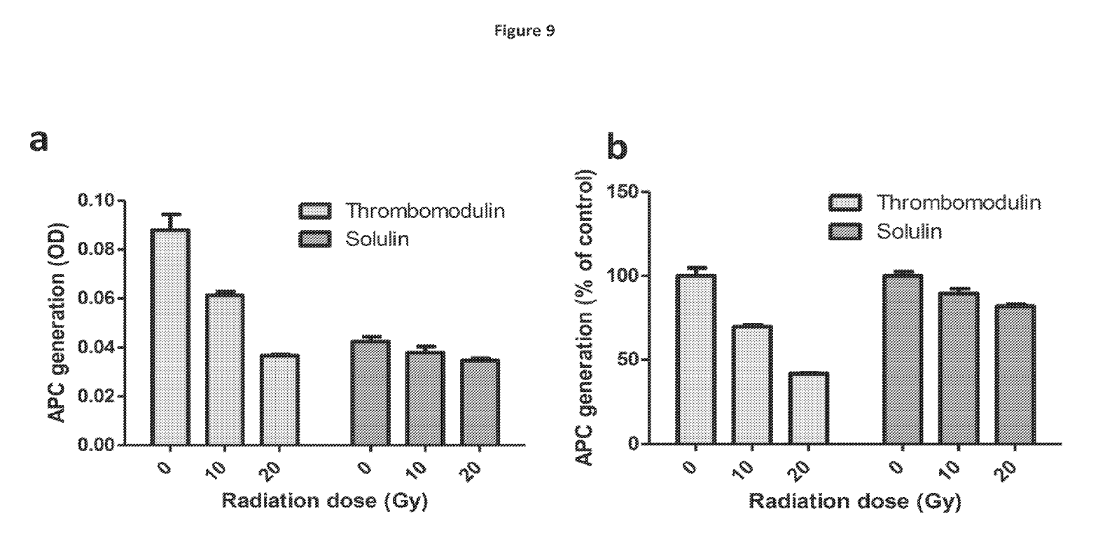

Administration of an oxidation-resistant form of soluble, recombinant human THBD (solulin, INN sothrombomodulin alpha; see FIG. 9) up to 30 minutes post-TBI at 8.5 or 9.5 Gy resulted in significant radioprotection of wild-type mice, compared to vehicle-treated controls, with a 40%-80% survival benefit (FIGS. 2A-B). Solulin has been shown to serve as the cofactor for conversion of the plasma zymogen protein C (PC) into the natural anticoagulant, activated protein C (aPC). Wang et al., J. Biol. Chem. 275:22942-22947 (2000); van Iersel et al., Thromb Haemost. 105:302-312 (2011); Foley et al., J. Thromb. Haemost. 9:284 (2011). To determine whether the protective effects of soluble THBD could be related to the activation of protein C, it was investigated whether infusion of recombinant aPC could reproduce the radio-protective effect of soluble THBD.

In independent experiments conducted in three different laboratories, administration of recombinant murine aPC to C57BL/6 mice (at 5 μg/mouse i.v., equal to 0.4 mg kg⁻¹) conferred a significant survival benefit compared to vehicle-treated controls (FIGS. 2C-D). Similar data were obtained with genetically distinct CD2F1 mice (at 0.35 mg kg⁻¹ i.v., 30 minutes post-TBI, data not shown), indicating that the aPC effect was not dependent on genetic background. At 10 Gy, still 40% of animals survived after multiple injections of aPC (at 30 minutes, 1 hour, and 2 hours post-TBI) (FIG. 2D). Remarkably, even when the first injection of aPC was delayed until 24 hours post-TBI, with a second injection at 48 hours post-TBI, significant radiation mitigation was observed (FIGS. 2E-F).

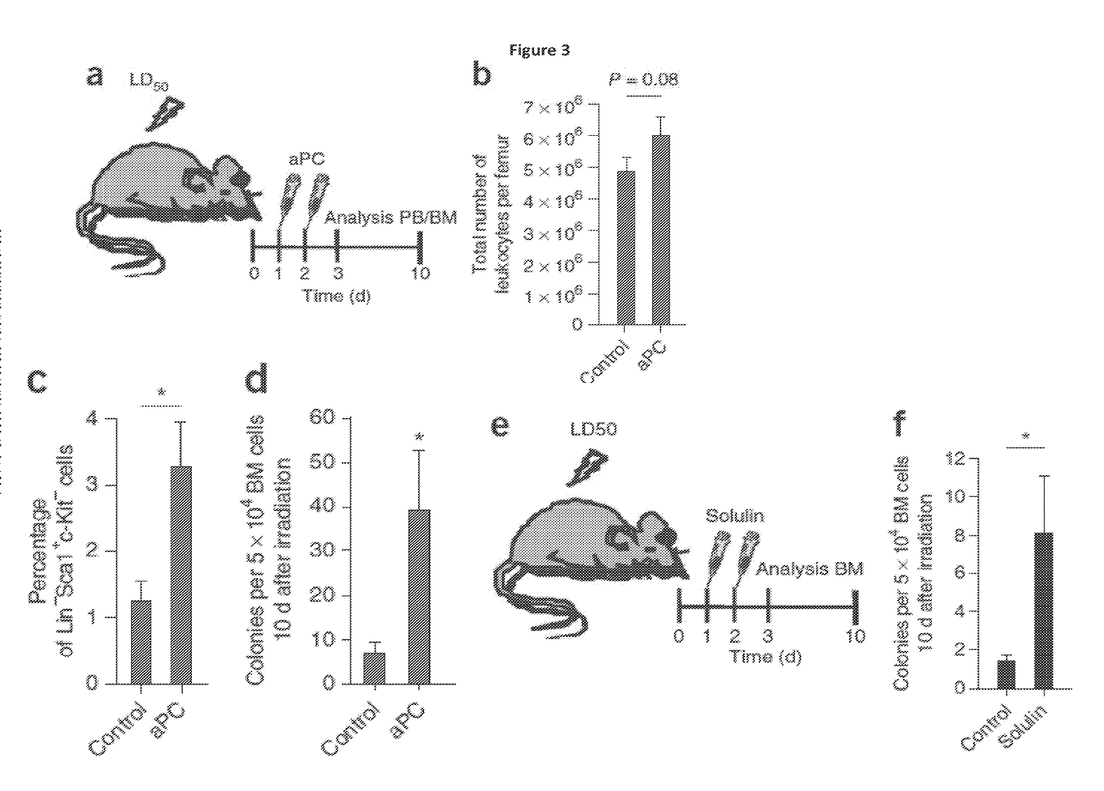

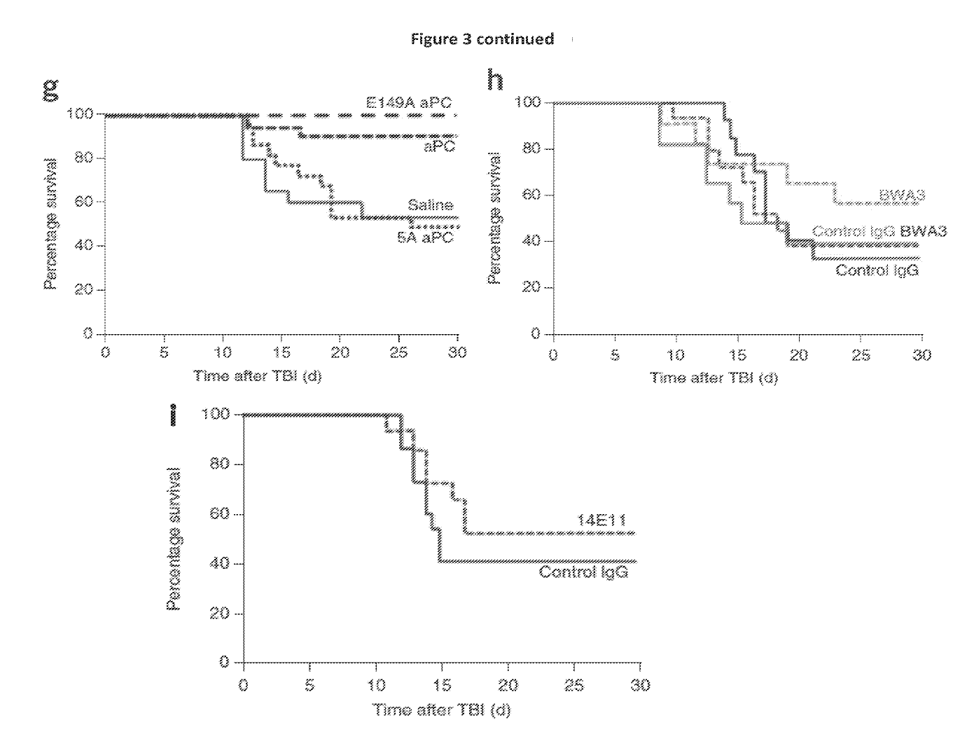

Given that the radiation doses used in our experiments result in death occurring 12-20 days post radiation exposure primarily due to failure of hematopoietic cells within the bone marrow, likely candidate cellular targets of solulin as well as aPC in radiation mitigation include epithelial and/or endothelial structures of the gut or bone marrow or bone marrow hematopoietic cells. Administration of solulin or aPC to lethally irradiated mice had no detectable effect on basic blood cell parameters at day 3 or 10 after radiation exposure (FIG. 10A, data not shown), except for marginally elevated numbers of white blood cells in BM at day 10 in aPC treated animals (FIGS. 3A-B). Hematopoietic progenitor cells in BM, determined by flow cytometry (Lin⁻, c-Kit⁺ cells, FIG. 3C) or functionally via CFU-C assays (FIG. 3D), were almost undetectable at 3 days post-irradiation (data not shown), but at 10 days the were significantly increased in aPC-treated mice compared to controls. The frequency of animals presenting with more than 10 CFU-C colonies per 5×10⁴ cells in BM at day 10 post TBI was likewise significantly higher in the aPC-treated group (data not shown), and correlated with the frequencies of aPC-treated animals surviving exposure to lethal radiation doses (see FIGS. 2D, 2F). Similar observations were made in lethally irradiated mice receiving solulin (FIGS. 3E-F), consistent with the notion that activation of protein C constitutes a relevant downstream effector of soluble THBD. Infusion of aPC did not alter biomarkers of radiation-induced gut injury, such as plasma citrulline levels and integrity of the gut epithelial surface (FIGS. 10C-E), indicating that mitigation of radiation damage to the intestine was unlikely to significantly contribute to the efficacy of aPC.

To gain more insight on molecular mechanisms of aPC action, we compared distinct recombinant variants of aPC with respect to their radio-mitigating activity. The mouse 5AaPC variant, which exhibits full Eper- and Par1-mediated cytoprotective function, but only residual (˜8%) anticoagulant activity (Mosnier et al., J. Biol. Chem. 282:33022-33 (2007); Kerschen et al., J. Clin. Invest. 120:3167-78 (2010)) did not prevent radiation-induced mortality. In contrast, infusion of E149A-aPC with augmented anticoagulant activity but with deficient signaling activities, (e.g., only ˜5% of normal anti-apoptotic activity) (Mosnier et al., Blood 113:5970-8 (2009)) conferred a significant survival benefit that was comparable to that of wild type aPC (FIG. 3G). The biological activity of aPC that mediates radiomitigation is thus preserved in the E149A-aPC variant, but compromised in the 5A-aPC variant.

Both Par1 and Eper are expressed on primitive BM cells. See, e.g., Ramalho-Santos et al., Science 298:597-600 (2002); Kent et al., Blood 113:6342-6350 (2009); Balazs et al., Blood 107:2317-2321 (2009); Iwasaki et al., Blood 116:544-553 (2010). HSPC lacking Par1 (Darrow et al., Thromb. Haemost. 76:860-866 (1996)) or expressing greatly diminished levels of Eper (Iwaki et al., Blood 105:2364-2371 (2005)) were not impaired but rather slightly favored compared to wild type controls in competitive transplantation/radiation-injury experiments (FIGS. 11A-C), providing additional support that the radio-mitigation activity of aPC does not involve Eper and Par1-dependent signaling on HSPCs which is consistent with the failure of the signaling-selective 5A-aPC variant to afford radio-protection.

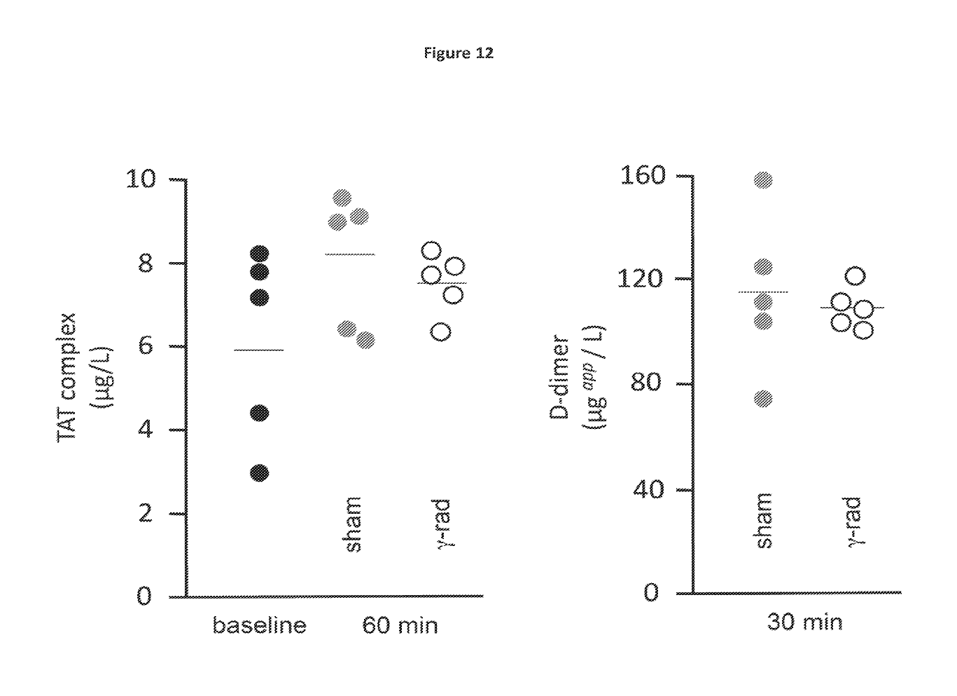

Functions preserved by the E149A-aPC variant, but deficient in the 5A-aPC molecule potentially include the anticoagulant effect of aPC and potentially coagulation-independent aPC effects, such as the degradation of cytotoxic histone-DNA complexes released from damaged cells. Xu et al., Nat. Med. 15:1318-21 (2009). However, inhibition of cytotoxic histones 3 and 4 with the function blocking BWA3 antibody, using conditions shown previously to reduce mortality in sepsis (Xu et al., Nat. Med. 15:1318-21 (2009); Xu et al., J. Immunol. 187:2626-2631 (2011)) did not result in radioprotection (FIG. 3H). Similarly, inhibition of the intrinsic coagulation pathway with anti-fXI antibody 14E11 (Luo et al., Infect Immun. 80:91-99 (2012)) (FIG. 3I) or with low molecular weight heparin were both ineffective in radioprotection. Biomarkers indicative of the activation state of the blood coagulation system in PB (plasma thrombin/anti-thrombin complexes and fibrin D-dimer) were unaltered over a 24 hour window of time following exposure to lethal radiation doses (FIG. 12), suggesting that the radioprotective effect of the E149A-aPC variant is not due to its anti-thrombotic actions. While it is possible that optimized dosing with antibodies blocking thrombosis or histone-induced inflammation might reveal some beneficial effect of these reagents on radiation injury, it seems unlikely that the pathological mechanism inhibited by these antibodies are the critical targets of aPC that mediate the accelerated recovery of HPC activity and survival of radiation injury. The targets of aPC mediating accelerated recovery of HPC activity and survival of radiation injury therefore remain presently unknown and likely involve novel functions associated with wild-type aPC and the E149A-aPC variant. Mosnier et al., Blood 113:5970-5978 (2009); Kerschen et al., J Exp Med. 204:2439-2448 (2007).

### Example 3

### Thrombomodulin (Thbd)-Activated Protein C (aPC) and Radiation Mitigation

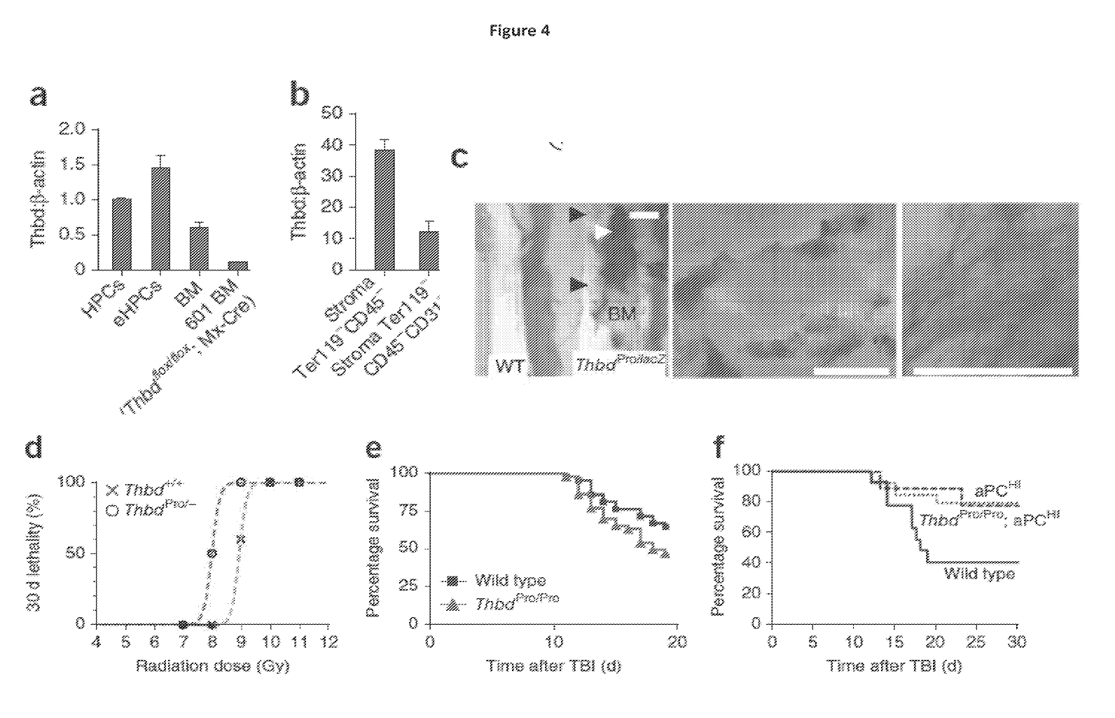

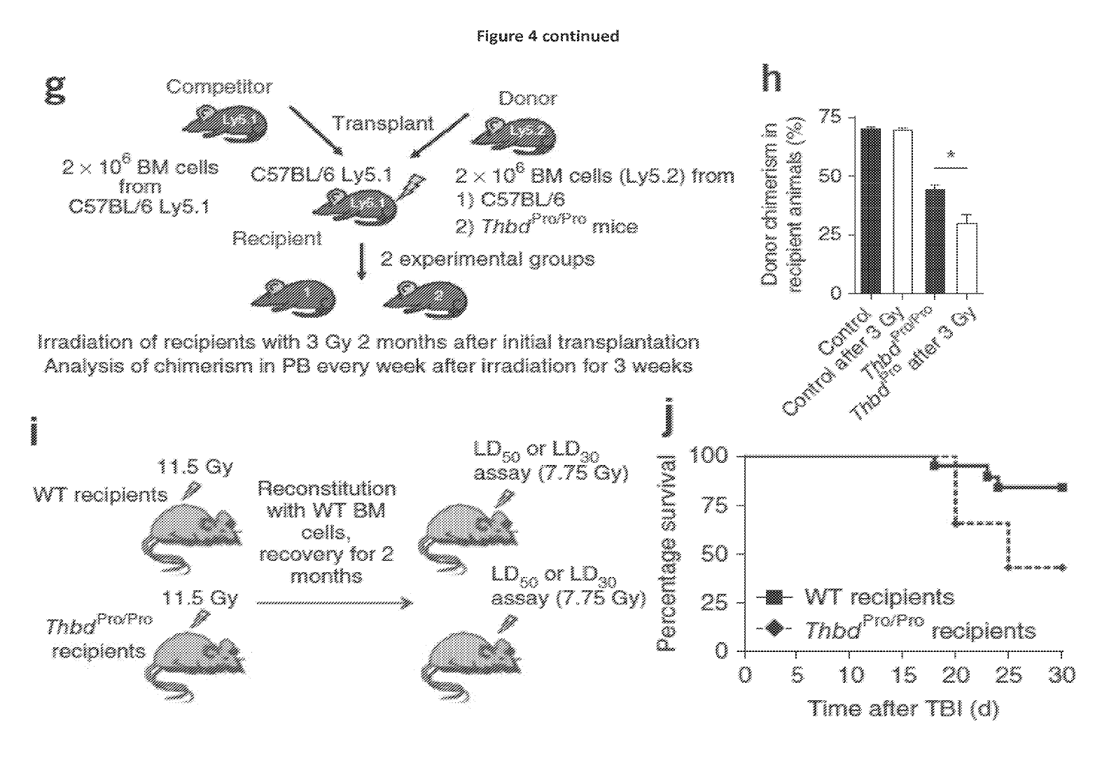

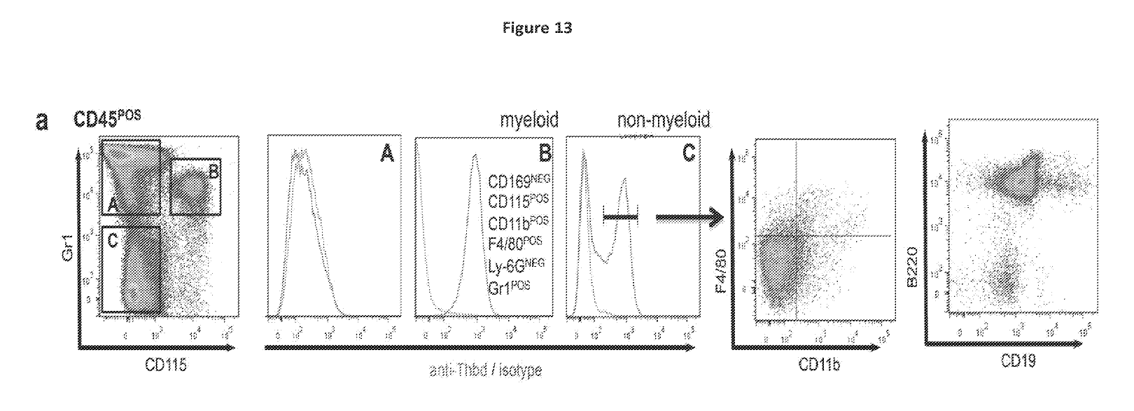

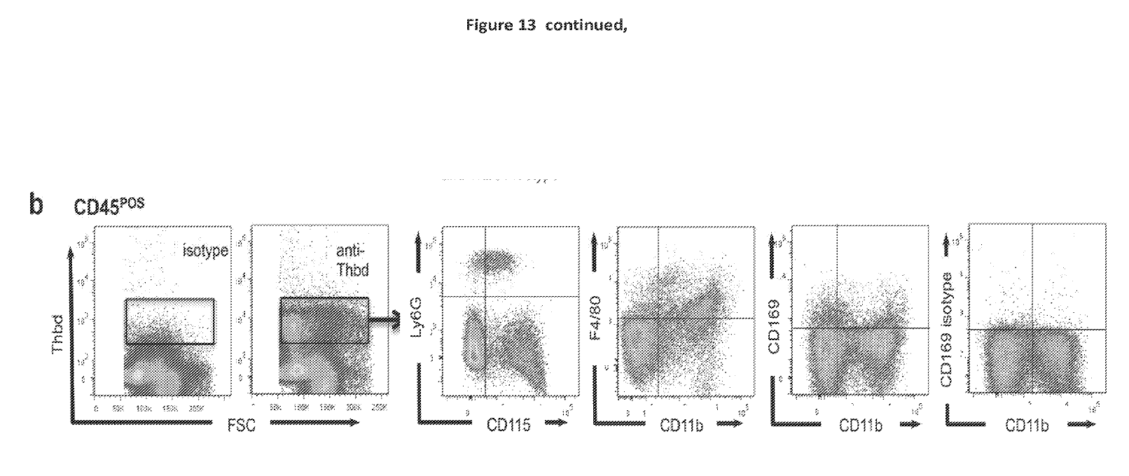

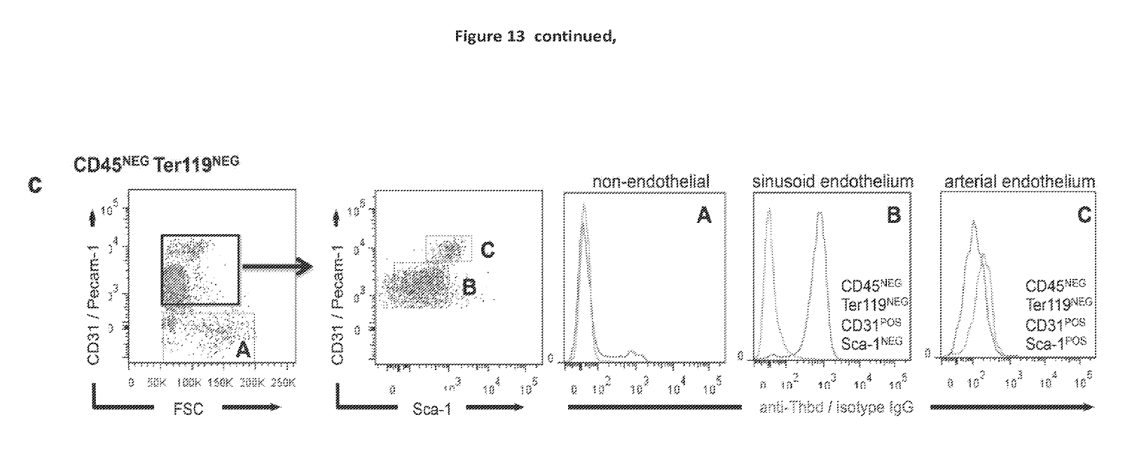

The above-described data raise the question whether the endogenous Thbd-PC pathway plays a previously unrecognized role in mitigating the lethal consequences of radiation-induced bone marrow failure. In adult mice and humans, Thbd is expressed ubiquitously in endothelial cells of small blood vessels except for low levels in certain brain microvascular beds. Ismail et al., Cardiovasc. Pathol. 12:82-90 (2003). Within the human hematopoietic system, THBD is expressed in a subpopulation of human dendritic cells, in monocytes, and in a small subset of neutrophils. Conway et al., Blood 1992; 80:1254-63 (1992); Yerkovich et al., J. Allergy Clin. Immunol. 123:209-216 (2009). Western Blot analysis of BM from radiation-exposed animals indicated the presence of Thbd protein in BM cells (FIG. 1D, GFP⁻ control) and Thbd transcript was detected in differentiated BM cells, in HPCs, in eHPCs (Lin⁻/c-Kit⁻/Sca-1⁺ cells) of BM (FIG. 4A), in BM-derived CD45⁻/Ter111⁻/CD31⁺ endothelial cells, and in the CD45⁻/Ter111⁻/CD31⁻ stroma cell compartment in BM (FIGS. 4A-B). An in situ survey of β-galactoside expression in the femur of Thbd^{lacZ} knock-in mice indicated abundant Thbd expression within the endosteal region, as well as in femoral blood vessel endothelial cells supplying the marrow (FIG. 4C). Flow cytometry confirmed Thbd expression in Ly-6G^{NEG}Gr1^{POS}CD115^{POS} BM macrophages as well as in B220/CD19 positive B-cell precursors (FIG. 13A; additional data not shown). Thbd-expressing macrophages are distinct from the two previously described populations of BM resident macrophage-like cells involved in maintenance of the hematopoietic niche in BM, i.e., cells with the surface phenotype CD169^{POS}/CD115^{INT}/F4-80^{POS}/Gr1^{NEG} or CD11b^{POS}/Ly-6G^{POS}/F4-80^{POS} (FIG. 13B). Within the CD45^{NEG}/Pecam-1/CD31^{POS} endothelial population in BM, Thbd expression is detected in Sca-1^{NEG} sinusoidal endothelium, but is absent from Sca-1^{POS} arterial endothelium (FIG. 13C). This combined analysis of Thbdm RNA, Thbd-antigen, and lacZ-reporter gene expression is consistent with the presence of Thbd on hematopoietic cells and non-hematopoietic cells within the bone marrow.

It was next investigated whether the selective genetic disruption of protein C activation by endogenous Thbd which results in minimal residual Thbd function modified TBI survival. Weiler-Guettler et al., J. Clin. Invest. 101:1983-1991 (1998); Weiler-Guettler et al., Arterioscler. Thromb. Vasc. Biol. 21:1531-1537 (2001); Weiler-Guettler et al., Circ. Res. 78:180-187 (1996). Mice (Thbd^{Pro/LacZ}) carrying only one functional Thbd-allele encoding a Thbd variant (Thbd^{Pro}) with severely reduced ability to activate protein C (Weiler-Guettler et al., Arterioscler. Thromb. Vasc. Biol. 21, 1531-1537 (2001)) showed increased sensitivity to TBI, with the dose of radiation eliciting 50% lethality shifted from about 8.75 Gy in wild-type mice to about 7.5 Gy in Thbd-deficient mice (FIG. 4D), while Thbd^{Pro/Pro} mice, which show a less severe Thbd deficiency than Thbd^{Pro/LacZ} mice still presented with elevated sensitivity to TBI (FIG. 4E). In contrast, aPC^{HI} transgenic mice having constitutively elevated plasma aPC levels due to expression of a variant human protein C that is efficiently activated by thrombin even in the absence of Thbd (Isermann et al., Nat. Med. 13, 1349-58 (2007)) were protected against radiation induced BM failure to a similar extent as wild-type mice treated with recombinant aPC (FIG. 4F). Expression of the aPC^{HI} transgene also rescued the increased radiation sensitivity of Thbd-deficient Thbd^{Pro/Pro} mice (FIG. 4F), providing direct genetic evidence that the increased radiation sensitivity of Thbd-deficient mice is caused by inadequate activation of endogenous PC.

Competitive hematopoietic reconstitution of lethally irradiated wild-type recipients with BM from Thbd^{Pro/Pro} and wild-type mice, followed by exposure to 3 Gy TBI given 8 weeks after transplantation (FIG. 4G), showed a significantly reduced recovery of Thbd^{Pro/Pro} cells when compared to wild-type cells (FIG. 4H). The initially lower contribution of Thbd^{Pro/Pro} bone marrow cells to chimerism before irradiation might be caused by additional functions of Thbd in HSC biology that have not yet been investigated. Non-competitive reconstitution of irradiated Thbd^{Pro/Pro} recipients with wild-type bone marrow (FIG. 4I), followed by a second exposure to a LD₅₀-dose of TBI, resulted in significantly increased 30 day mortality compared to wild-type animals reconstituted using wild-type BM (FIG. 4J). Hence, endogenous Thbd expression on hematopoietic cells as well as on non-hematopoietic stroma cells affords protection against radiation mirroring the protection conferred by forced Thbd overexpression in HSPC or by therapeutic administration of soluble Thbd/aPC.

The experiments described herein identified the Thbd-protein C pathway as a physiologically relevant mechanism for accelerating HSPC recovery in response to lethal TBI to an extent that results in significant radiomitigation. In addition, the data presented herein are consistent with a mechanism in which endogenous Thbd expression on stromal endothelial cells promotes protein C activation and release of aPC into the BM microenvironment, followed by aPC-stimulated recovery from radiation-induced hematopoietic suppression. As indicated by the effect of Thbd deficiency on 30-day post-TBI mortality (FIG. 4J), the contribution to overall survival provided by stromal endothelial expression of Thbd expression is apparently more important than hematopoietically-expressed Thbd. Nevertheless, Thbd expression on HSPC supports hematopoietic recovery upon TBI in an apparent cell-autonomous manner, with as yet unknown effects on whole animal survival of radiation injury. While the cellular and molecular mechanisms underlying the effect of soluble THBD or aPC on the recovery of HPC activity requires further study, these mechanisms are likely distinct from previously explored pathways, including those mediated by agonists of toll-like receptor 5 (Burdelya et al., Science 320:226-230 (2008)), inhibitors of CDK4/6, or various antioxidant compounds (Johnson et al., J Clin Invest. 120:2528-2536 (2010)). While the effects of endogenous aPC are largely confined to the local microenvironment of Thbd-expressing cells, systemic administration of soluble THBD or of aPC can reproduce and augment the radioprotective effects of the endogenous Thbd-aPC pathway.

While the cellular and molecular mechanisms underlying the cytoprotective effects of aPC and aPC variant polypeptides remain elusive, it is known that the effects are not elicited by antithrombotic agents that inhibit fXI-dependent intrinsic coagulation pathways, nor are they elicited using histone inhibitory antibodies. Geiger et al., Nature Medicine 18(7):1123-29 (2012).

Recombinant human aPC has undergone extensive clinical testing in patients, and recombinant soluble human Thbd is being investigated as a therapeutic antithrombosis for clinical use. The data presented herein encourage further evaluation of these proteins for radiomitigating activity. These agents, possibly in combination with compounds targeting other pathways, may provide novel clinically relevant counter-measures to combat radiation induced pathologies resulting from environmental or therapeutic exposure to high levels of radiation.

Materials and Methods

Animals: Animals were housed under standardized conditions with controlled temperature and humidity and a 12/12-hour day/night light cycle. C57BL/6, C57BL/6CD45.1, and CD2F1 mice were obtained from commercial vendors (Charles River, The Jackson Laboratories, Harlan Sprague Dawley). Thbd^{Pro/Pro-}, Thbd^{lacZ-}, Eper^{low-}, and Par1^{-/-} mice have been described previously and were maintained on a C57BL/6 background (>14 backcrosses). See, e.g., Weiler-Guettler et al., J. Clin. Invest. 101:1983-1991 (1998); Weiler et al., Arterioscler. Thromb. Vasc. Biol. 21:1531-1537 (2001); Weiler et al., Thromb. Haemost. 92:467-477 (2004); Darrow et al., Thromb. Haemost. 76:860-866 (1996); Connolly et al., Nature 381:516-519 (1996); and Iwaki et al., Blood 105:2364-2371 (2005). Due to early embryonic lethality in Thbd-null mice, mice having minimal residual Thbd function were obtained. Such mice are Thbd^{Pro/lacZ}, carrying one Thbd^{Pro} allele that encodes a Thbd variant (Thbd^{Pro}) having a greatly diminished ability to support Protein C activation (Weiler-Guettler et al., J. Clin. Invest. 101:1983-1991, 1998; Weiler et al., Arterioscler. Thromb. Vasc. Biol. 21:1531-1537, 2001), and one Thbd^{null} allele for which the Thbd gene is disrupted by insertion of a lacZ reporter (Weiler-Guettler et al., Circ Res. 78:180-187, 1996). The Thbd^{Pro} mouse carries a point mutation resulting in a Glu-Pro exchange in the EGF3-4 inter-domain loop of Thbd. The mutation substantially suppresses Thbd-dependent aPC generation but retains additional functions associated with Thbd, such as lectin-domain effects on complement regulation and leukocyte-endothelial interaction. Thbd antigen levels in the lung are reduced 2-3-fold in Thbd^{Pro/Pro} mice. In all animal experiments, male and female mice were used in unspecified ratios. Animal experiments were approved by the IACUCs at Cincinnati Children's Hospital Medical Center (CCHMC), the University of Arkansas for Medical Sciences (UAMS)/Central Arkansas Veterans Healthcare System (CAVHS), or the Medical College of Wisconsin.

Reagents: Recombinant human Thbd (CD141) (Cat no. 2374) and PC (Cat. no. 239F) for in vitro experiments (protein C activation assay) were purchased from American Diagnostica. Thrombin was purchased from Sigma. Recombinant human Thbd (CD141) (10 mg) was purified from a Chinese Hamster Ovary (CHO) cell line and provided as a lyophilized powder that was reconstituted with deionized water. aPC (100 μg/ml) was provided in a 50% (vol/vol) glycerol/water mixture. Recombinant mouse wild-type aPC, a “signaling selective” aPC variant (5A-aPC), and a “signaling-defective” aPC variant (E149A-aPC, a hyper-anti-thrombotic, non-cytoprotective Glu149Ala mutant version of aPC) have been described previously (Mosnier et al., Blood 113:5970-5978, 2009; Mosnier et al., J. Biol. Chem. 282:33022-33, 2007). Solulin (soluble human recombinant thrombomodulin, ZK 158 266, INN sothrombomodulin alfa), which is the modified recombinant human Thbd molecule (Paion Deutschland GmbH), comprises the extracellular portion of Thbd (the N-terminal lectin-binding domain, six EGF-like repeats, and the serine/threonine-rich domain), but lacks the transmembrane and intracellular domains, as well as the chondroitin sulfate moiety. It is derived from the molecule originally described by Glaser et al. (J. Clin. Invest. 90:2565-2573, 1992) and referred to as TMLEO10, but includes the following mutations: deletion of the first three N-terminal amino acids, Met388Leu, Arg456Gly, His457Gln, Ser474Ala, and deletion of the last seven C-terminal amino acids. amino acids of the carboxy terminus (Weisel et al., J Biol Chem. 271:31485-31490, 1996). Solulin was provided as a sterile liquid solution for injection comprising Solulin (4.6 mg/ml) in 10 mM sodium phosphate, 2.7 mM potassium chloride, 137 mM sodium chloride, and 5% mannitol at pH 7.0.

Hybridoma cells secreting the rat anti-mouse Thbd monoclonal AB 273-34A and 411-201B 12 were generously provided by Dr. Kennel (University of TN Graduate School of Medicine, Knoxville, Tenn., USA). The AB was used at a 1:200 dilution. For flow cytometric analyses, a polyclonal secondary anti-rat antibody labeled with APC was used for detection of Thbd expressing cells. The following antibodies were also used: Thbd (M-17) (an affinity purified goat polyclonal antibody; sc-7097, Santa Cruz Biotechnology); BWA3 antibody (Temple University School of Medicine, Philadelphia, Pa.); 14E11 antibody (Vanderbilt University, Nashville, Tenn.).

Total Body Irradiation (TBI) Protocol: Mouse TBI was performed at the CAVHS with a Shepherd Mark I-25 Cs-137 irradiator (J.L. Shepherd & Associates) as described (Weisel et al., supra). The average dose rate was 1.37 Gy per minute. In some cases, irradiation was performed with a Shepherd Mark I-68 Cs-137 irradiator with an average dose rate of 0.52 Gy per minute (located at CCHMC), or a Gammacell 40 Extractor Cs-137 (Best Theratronics; average dose rate 0.97 Gy-min⁻¹, Blood Research Institute). All irradiators were calibrated annually. For TBI mitigation experiments, the mice were monitored for up to 30 days post-TBI. The number of dead/moribund mice was recorded twice daily. Kaplan-Meier survival curves, median survival times, and lethality at 30 days were recorded.

Retroviral and Lentiviral Transduction of Hematopoietic Cells:

Hematopoietic stem and progenitor cells (HSPCs) derived from bone marrow were transduced with a SFbeta virus containing an IRES EGFP sequence or a Thbd IRES EGFP construct at a multiplicity of infection of about 2-4, which results on average in 1 or 2 integration sites per single genome. These cells were subsequently transplanted into recipient animals preconditioned with 11.75 Gy (7 Gy+4.75 Gy, 4 hours apart), as previously described (Modlich et al., Blood 108:2545-53, 2006; Kustikova et al., Blood 109:1897-1907, 2007; Kustikova et al., Mol. Ther. 17:1537-1547, 2009). LM-PCR and integration site sequencing were performed as previously described (Kustikova et al., Blood 109:1897-1907, 2007).

Blood Parameters, Flow Cytometry, Isolation of Stroma Cells, and Quantitative Real-Time RT-PCR:

Blood parameters were determined by a Hemavet Instrument (Drew Scientific Inc). Immunostaining and flow cytometry analyses were performed according to standard procedures and analyzed on a FacsCanto flow cytometer (BD Biosciences). Anti-Ly5.2 (clone 104, BD Biosciences, FITC conjugated) and anti-Ly5.1 (clone A20, BD Biosciences, PE conjugated) monoclonal antibodies were used to distinguish donor cells from recipient and competitor cells. For lineage analysis in hematopoietic tissues, anti-CD3ε (clone 145-2C11, PE-Cy7 conjugated), anti-B220 (clone RA3-6B2, APC conjugated), anti-CD11 b (clone M1/70, APC-Cy7 conjugated), and anti-Gr-1 (clone RB6-8C5, APC-Cy7 conjugated) (all from BD Biosciences) were used. Antibodies were used at a 1:100 dilution. The level of RNA expression was determined by real-time RT-PCR using Taqman Universal PCR and RT reagents from ABI (Life Technologies Corporation). RNA was purified using a Qiagen RNeasy micro kit (Qiagen Inc). Table 1 presents Taqman probes used to determine expression of selected genes in the neighborhood of the viral integration site in experimental animals.

TABLE 1
Taqman Gene Expression Probes

| Mouse ID | Gene Name | Ref Seq No. | Assay ID |
|---|---|---|---|
| Mouse 2 | Bbc3 (Puma) | NM_133234.1 | Mm00519268_m1 |
| | Ccdc9 | NM_172297.1 | Mm00552307_m1 |
| | Ubel1a | NM_019748.1 | Mm00502282_m1 |
| Mouse 7 | Obfc1 | NM_175360.2 | Mm00614838_m1 |
| | Junb | NM_008416.1 | Mm00492781_s1 |
| | Hook2 | NM_133255.1 | Mm00466969_m1 |
| | Ly86 | NM_010745.1 | Mm00440240_m1 |
| | Rreb1 | NM_026830.1 | Mm01325346_m1 |
| Mouse 9 | Sla | NM_001029841.1 | Mm00447294_m1 |
| | Thbd | NM_009378.1 | Mm00437014_S1 |
| | CD93 | NM_010740.2 | Mm00440239_g1 |

Assessing Effects of TBI:

To analyze the effect of TBI on Thbd^{Pro/lacZ} animals, mice were subjected to TBI at 7 Gy or 11 Gy. For competitive transplants followed by TBI for radioselection, BM cells were harvested from tibia and femur of 6- to 8-week-old Thbd^{Pro/Pro} mice (donor) as well as B6.SJL (BoyJ) (competitor) mice (2×10⁶ cells from each) and transplanted into BoyJ recipient mice that had been lethally irradiated with a total dosage of 11.75 Gy (7 Gy+4.75 Gy, 4 hours apart). Cells were transplanted into the retro-orbital sinus or via tail vein in a volume of 200 μL in IMDM/2% FCS. Three to four weeks post-transplantation, peripheral blood chimerism was analyzed using flow cytometry with a panel consisting of CD45.2, B220 for B cells, CD3ε for T cells, and Mac-1/Gr-1 combined for the myeloid lineage. Animals were subsequently irradiated with 3 Gy TBI for radioselection, and peripheral blood was analyzed for chimerism at the time-points following 3 Gy indicated in the experiments.

Assessing Effects of Recombinant Polypeptides:

Mice were subjected to TBI at the doses indicated and subsequently randomly assigned to receive wild-type aPC, a signaling-selective aPC (5A-aPC), a signaling-defective aPC (E149AaPC, a hyperantithrombotic, non-cytoprotective Glu149Ala aPC mutant), or vehicle. aPC or its variants were dissolved in vehicle buffer (50 mM Tris, 100 mM NaCl, pH 7.4). Mice received a single dose or, where indicated, multiple doses of either recombinant aPC, an aPC variant, or vehicle buffer at 0.35 to 0.4 mg/kg in a volume of 200 μL or less by tail vein (i.v.) injection at the time points indicated after TBI. Male mice were injected subcutaneously with Solulin (1 mg/kg or 3 mg/kg) or vehicle 30 minutes post-TBI. The mice were returned to the cages with free access to food and water and monitored twice daily for 30 days for weight loss, apparent behavioral deficits, and survival.

Apoptosis/Survival Assay:

Transduced cells sorted for EGFP were subjected to 1.5 Gy in vitro and cultured overnight in Iscove's modified Dulbecco's medium supplemented with 10% FBS, 2% penicillin-streptomycin, 100 ng/ml of mSCF, 100 ng/mL of TPO and 100 ng/mL of G-CSF. At 18 hours post-irradiation, cells were harvested and washed twice with ice cold PBS and 1×10⁵ cells were resuspended in PBS. Cells were stained with anti C-kit-APC antibody (BD Pharmingen) for 20 minutes on ice. The cells were then stained with anti-Annexin V PE antibody (BD Pharmingen) and 7AAD (1 mg/mL) in 1× binding buffer and analyzed using a BD FACSCanto. Antibodies were used at a 1:100 dilution.

Proliferation Assay:

Triplicates of 2-5×10⁵ transduced cells were resuspended in 2 ml of IMDM supplemented with 10% FBS, 2% penicillin-streptomycin, 100 ng/mL of murine stem cell factor, 100 ng/mL of TPO, and 100 ng/mL of granulocyte colony-stimulating factor. Cell counts were determined on day 3, 5, and 7.

Colony Forming Assay:

CFC assays were performed using methocult (M3234 Stem Cell Technologies Inc) containing a final concentration of 50 ng/mL mSCF, 10 ng/mL mIL-3, and 10 ng/mL mIL-6 (Peprotech). The cells were plated in triplicates in 6-well plates and irradiated with 1.5 or 3 Gy using a Cs-137 source. Plates were incubated at 37° C. in 5% CO₂ and colonies were counted between 7 and 10 days after plating.

In Vitro Irradiation:

In vitro irradiation experiments were performed in a cell-free system (i.e., in a system not confounded by transcriptional regulation or ectodomain shedding of Thbd) as described previously (Ross et al., Radiat. Res. 169:408-416, 2008). Briefly, all Thbd and Solulin samples were dissolved in buffer (described herein) and irradiated in 1-mL polypropylene microcentrifuge tubes in a total volume of 500 μL. At least 3 independent samples for Thbd and Solulin were irradiated at each dose level for all experiments, not including optimization and validation studies.

Assaying Thbd Activity:

Recombinant Thbd and Solulin were dissolved to a final concentration of 50 nM in a buffer comprising 10 mM Tris-HCl, 0.2 M NaCl, 5 mM CaCl₂, and 0.1% polyethylene glycol, pH 8.0. The effects of single-dose irradiation on Thbd functional activity were assessed using a protein C activation assay following exposure to 0, 1.77 Gy (0.3 min radiation exposure), 10 Gy (1.7 min), 20 Gy (3.4 min), and 40 Gy (6.8 min). PC activation assays were performed as follows: irradiated and sham-irradiated samples were diluted to a final concentration of 2.5 nM Thbd and incubated with 200 nM PC and 1.4 nM thrombin for 60 minutes at 37° C. in a 96-well plate to generate aPC. The amount of aPC generated was measured by monitoring hydrolysis of the chromogenic substrate, S-2366 (Diapharma), at 405 nm in a microplate reader (Bio-Tek Instruments) at 5-minute intervals for 60 minutes. The results were expressed as the mean optical density (OD) at 60 minutes. All assays were performed in triplicate, and the average was considered a single value for statistical purposes.

Solulin Pharmacokinetics:

As intravenous pharmacokinetics of Solulin in mice and rats are highly comparable (PAION Deutschland GmbH, data not shown), dose selection for subcutaneous administration of Solulin in mice was based on available rat data. Solulin, injected subcutaneously at 3 mg/kg in male rats, reached plasma concentrations of about 40 nM after 6 hours and about 100 nM after 26 hours (maximal observed value). As these levels are associated with pronounced aPC-mediated effects of intravenous Solulin in animal models of thrombotic vessel occlusion (see, for example, Solis et al., Thromb. Res. 73:385-394, 1994) doses of 1 and 3 mg/kg were selected for mouse radiation studies.

Measuring Plasma Citrulline Levels:

The plasma level of citrulline is a well-validated biomarker for functional enterocyte mass (Crenn et al., Clinical Nutrition 27:328-339 (2008)) and exhibits close correlations with other markers of intestinal radiation injury including mucosal surface area and the crypt colony assay. Lutgens et al., Int J. Radiat. Oncol. Biol. Phys. 57:1067e74 (2003); Berbée et al., Radiation Research 171(5):596-605 (2009). Plasma citrulline levels reflect enterocyte mass and correlate directly with radiation dose. Plasma citrulline concentration was determined using an LC-MS/MS as previously described (Gupta et al., Analytical Methods 3:1759-1768 (2011)). Briefly, plasma proteins were precipitated in a 96-well Strata Impact 2 ml filtration plate (Phenomenex, Torrance, Calif.). Each plasma sample (10 μL) was treated with 490 μL acetonitrile::water::formic acid (85:14.8:0.2 v/v) containing internal standard (2 μM). After mixing gently, the plate was covered, allowed to stand for 5 minutes, and the filtrate was collected under vacuum. Calibration was performed using an isotope dilution approach where L-citrulline served as a calibration standard (0.125-200 μM) and L-Citrulline (5-13C, 99%; 4,4,5,5-D4, 93%+) (“citrulline+5”) as an internal standard. The LC-MS/MS system was an Acquity UPLC system interfaced to a Quattro Premier triple quadrupole mass spectrometer (Waters Corp.). Chromatographic separation was achieved on a Phenomenex 1.7 μM Kinetex HILIC analytical column (50×2.1 mm (i.d.)). Mobile phase A was acetonitrile containing 0.1% formic acid, 0.2% acetic acid and 0.005% trifluoroacetic acid. Mobile phase B was water containing 0.1% formic acid, 0.2% acetic acid and 0.005% trifluoroacetic acid. The initial flow rate was 0.6 ml/min and then increased to 0.7 ml/min at 1.3 min. The gradient program parameters were as follows: initial 9% B; 0-1.2 min: 11% B; 1.2-1.3 min: 30% B and held for 0.6 min; 1.9-2.0 min: 9% B. The total run time was 3.0 minutes. The sample injection volume was 3 μL. The sample loop volume was 10 μL. Positive ions for citrulline and citrulline+5 were generated using electrospray ionization at a capillary voltage of 3 kV and cone voltage of 18 V. Product ions were generated using argon collision induced disassociation at collision energy of 10 eV while maintaining a collision cell pressure of 4.8×10⁻³ torr. Mass spectrometric detection was performed in the multiple-reaction monitoring (MRM) mode using the precursor→product ions, m/z 176→159 and m/z 181→164 for citrulline and citrulline+5, respectively. The lower limit of quantitation was 0.125 μM while the upper limit of quantitation was 200 μM. Predicted values for all calibrators were within 90-110% of their nominal values.

Measuring Mucosal Surface Area:

Intestinal mucosal surface area is a validated and sensitive parameter of intestinal radiation injury. Sensitivity is greatest at the relatively low radiation doses such as those used in the methods described herein (i.e., less than 10 Gy) than for traditional crypt colony assays. Mucosal surface area was measured in vertical sections of the jejunum stained with hematoxylin and eosin (H&E), using a projection/cycloid method as previously described (Baddeley et al., J. Microsc. 142:259-276, 1986). We previously validated the method specifically for determining a surface area of intestinal mucosa after irradiation (Langberg et al., Radiother. Oncol. 41:249-255, 1996).

HSPC Staining:

2 million TBM cells were blocked with 2% mouse serum (M5905-5 ml, Sigma) for 10 minutes and stained using a mixture of biotin-conjugated lineage antibodies including CD5 (BD Biosciences Clone 53-7.3), CD45R (BD Biosciences, B220 Clone RA3-6B2), Mac1 (BD Biosciences, CD11b Clone M1/70), Gr1 (Biosciences, Ly-6G and Ly-6c BD Clone RB6-8C5), CD8a (BD Biosciences Clone 53-6.7) and Ter119 (BD Biosciences Clone TER-119) for 20 minutes. Cells were then washed once and stained with anti-cKit (BD Biosciences CD117 Clone 2B8, FITC conjugated), anti-Sca-1 (e Biosciences Ly-6A/E PECy7-Conjugated), and SA-APC-Cy7 (BD Biosciences 554063) for 30 minutes and analyzed using BD Facs Canto. Antibodies were used at a 1:100 dilution.

Statistical Methods:

Sample size for the TBI experiments was estimated as previously described (Kodell et al., Biometrics 66:239-248, 2010) for radiation countermeasure studies. Statistical analyses were performed using NCSS 2004 for Windows (NCSS). Data were presented as the mean±the standard error of the mean (SEM). Two-sided tests were used throughout, and differences were considered statistically significant when the p-value was less than 0.05. Pairwise (univariate) comparisons were performed with the Student's t-test or the Mann-Whitney U test as appropriate. Mouse survival curves were constructed using the Kaplan-Meier method, and 30-day survival rates were compared with logistic regression analysis within GraphPad Prism (Graphpad Sofware). The LD₅₀ values for comparison of mouse survival after radiation exposure were calculated with logit-transformed data, and standard error estimates were obtained by the delta (Δ) method.

We claim:

1. A method of treating or preventing a radiation injury in a subject, wherein the method comprises administering to the subject an effective amount of a polypeptide selected from Activated Protein C (APC), an APC variant, Plasma Zymogen Protein C (PC), and a PC variant, or a fragment thereof, wherein the amount is effective for treating or preventing radiation injury in the subject.

2. The method of claim 1, wherein the effective amount of the polypeptide is smaller than an amount of a wild-type APC polypeptide or PC polypeptide that treats or prevents a radiation injury.

3. The method of claim 1, wherein the effective amount is administered to the subject in a single or multiple bolus dose or a continuous infusion.

4. The method of claim 3, wherein the single dose or the two or more doses comprise a bolus comprising about 20 to about 600 microgram/kg APC polypeptide, about 20 to about 6000 microgram/kg APC polypeptide variant, or about 200 to about 50,000 microgram/kg of PC polypeptide or PC polypeptide variant.

5. The method of claim 1, wherein the APC variant comprises at least one amino acid modification relative to SEQ ID NO:1.

6. The method of claim 5, wherein the at least one amino acid modification comprises substitution of an alanine residue at position 149 or an alanine, aspartic acid, or glycine residue at position 191, 192, or 193, respectively, wherein amino acid residue positions are numbered relative to SEQ ID NO:1.

7. The method of claim 1, wherein the PC variant comprises at least one amino acid modification relative to SEQ ID NO:1.

8. The method of claim 7, wherein the PC variant is hyperactivatable.

9. The method of claim 8, wherein the hyperactivatable PC variant comprises a phenylalanine at amino acid residue position 167 and a lysine at amino acid residue position 172, wherein amino acid residue positions are numbered relative to SEQ ID NO:1.

10. The method of claim 1, wherein the radiation injury results from total body irradiation (TBI), ionizing radiation, or non-ionizing radiation.

11. The method of claim 10, wherein ionizing radiation is produced by cosmic radiation, nuclear medicine, an x-ray, nuclear fuel, or nuclear fallout.

12. The method of claim 1, wherein the radiation injury results from brachytherapy.

13. The method of claim 10, wherein only a portion of the subject is exposed to radiation.

14. The method of claim 1, further comprising administering an effective amount of a radioprotective drug to the subject.

15. The method of claim 14, wherein the radioprotective drug is γ-tocotrienol or CBLB502.

16. The method of claim 1, wherein the effective amount for treating or preventing radiation injury in the subject comprises an amount sufficient to yield a steady state plasma level in the subject of about 20 ng/mL to about 150 ng/mL APC polypeptide, about 20 ng/mL to about 800 ng/mL APC polypeptide variant, or about 20 ng/mL to about 12,000 ng/mL PC polypeptide or PC polypeptide variant.

17. The method of claim 1, wherein administering occurs immediately prior to, concurrently with, or following exposure of the subject to radiation.

18. The method of claim 1, wherein the subject is a human, a non-human animal, or a livestock animal.

19. A method of reducing chemical toxicity in a subject, wherein the method comprises administering to the subject an effective amount of a polypeptide selected from Activated Protein C (APC), an APC variant, Plasma Zymogen Protein C (PC), and a PC polypeptide variant, or a fragment thereof, wherein the amount is effective for treating or preventing radiation injury in the subject.

20. The method of claim 19, wherein the chemical toxicity results from administration of a myelosuppressive agent.

21. The method of claim 20, wherein the myelosuppressive agent is selected from the group consisting of cisplatin, carboplatin, 5-fluorouracil, bleomyocin, spiroplatin, marcellomycin, mitomycin C, doxorubicin, etoposide, cyclophosphamide, and bis-chloroethylnitrosourea (BCNU).

22. The method of claim 19, wherein the effective amount of the APC variant, the PC variant, or a fragment thereof, for treating or preventing radiation injury in the subject is smaller than an amount of APC polypeptide or PC polypeptide that reduces chemical toxicity.

23. The method of claim 19, wherein the APC polypeptide variant comprises at least one amino acid modification relative to SEQ ID NO:1.

24. The method of claim 23, wherein the at least one amino acid modification comprises substitution of an alanine residue at position 149 or an alanine, aspartic acid, or glycine residue at position 191, 192, or 193, respectively, wherein amino acid residue positions are numbered relative to SEQ ID NO:1.

25. The method of claim 19, wherein the PC variant comprises at least one amino acid modification relative to SEQ ID NO:1.

26. The method of claim 25, wherein the PC variant is hyperactivatable.

27. The method of claim 26, wherein the hyperactivatable PC variant comprises a phenylalanine at amino acid residue position 167 and a lysine at amino acid residue position 172, wherein amino acid residue positions are numbered relative to SEQ ID NO:1.

28. The method of claim 19, wherein the effective amount is administered to the subject in a single dose or in two or more doses.

29. The method of claim 28, wherein the single dose or the two or more doses comprise a bolus comprising about 20 to about 600 microgram/kg APC polypeptide, about 20 to about 6000 microgram/kg APC polypeptide variant, or about 200 to about 50,000 microgram/kg of PC polypeptide or PC polypeptide variant.

30. The method of claim 19, wherein the effective amount for treating or preventing radiation injury in the subject is an amount sufficient to yield a steady state plasma level in the subject of about 20 ng/mL to about 150 ng/mL APC, about 20 ng/mL to about 800 ng/mL APC variant, or about 20 ng/mL to about 12,000 ng/mL PC or PC variant.


SEQUENCE LISTING (1 sequence)

SEQ ID NO: 1 - protein, 419 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP